(19) United States
(12) Patent Application Publication
(10) Pub. No.: US 2006/0177478 A1
(43) Pub. Date: Aug. 10, 2006

(54) Intravascular blood conditioning device and use thereof

(76) Inventors: H. David Humes, Ann Arbor, MI (US); Evangelos Tziampazis, Plymouth, MI (US); Richard A. Andrews, Scotch Plains, NJ (US)

Correspondence Address: GOODWIN PROCTER LLP;PATENT ADMINISTRATOR, EXCHANGE PLACE, BOSTON, MA, 02109-2881, US

(21) Appl. No.: 11/388,876

(22) Filed: Mar. 24, 2006

Related U.S. Application Data
(63) Continuation of application No. 09/999,211, filed on Nov. 30, 2001.
(60) Provisional application No. 60/250,431, filed on Dec. 1, 2000.

(51) Int. Cl. A61K 9/22

(52) U.S. Cl. 424/423; 604/890.1

(57) ABSTRACT

Disclosed is an implantable, intravascular device for treating a medical disorder associated with the presence of a particular molecule in the systemic circulation of a mammal. When implanted, the device removes or reduces the concentration of the molecule in the blood stream thereby conditioning the blood. The device includes an anchor component immobilizable to an inner wall of an intact blood vessel and a cartridge component that is retained in place within the blood vessel by the immobilized anchor. The cartridge contains a converting agent, for example, viable cells or enzyme preparations, that catabolize or convert the molecule of interest into one or more other molecules that are not associated with the disorder. The invention also provides a minimally or non-invasive method for introducing into and, optionally, removing from the blood vessel the device of the invention.

### CROSS-REFERENCE TO RELATED APPLICATIONS

The present application claims priority to, and the benefit of U.S. Ser. No. 60/250,431, the entire disclosure of which is incorporated herein by reference.

### FIELD OF THE INVENTION

The present invention relates to an implantable, intravascular device for removing a pre-selected molecule from the blood stream of an animal, and to uses therefor. More particularly, the invention relates to an implantable, intravascular device which, when implanted into a blood vessel, removes or induces the removal of the pre-selected molecule from the blood stream, and to methods of using such a device.

### BACKGROUND OF THE INVENTION

Significant progress has been made in the identification of genetic and biochemical bases for a wide variety of mammalian disorders. In many cases, the disorder is caused by a deficiency of a particular molecule, for example, a hormone or enzyme. By using this type of knowledge, it has been possible to treat the disorder by replacing or supplementing the missing or deficient molecule. The molecule may be delivered either via direct administration, for example, by intravenous administration of the molecule or via indirect administration, for example, by administration of cells which synthesize and secrete the molecule into the recipient.

U.S. Pat. No. 4,378,016 describes a surgically implantable device for delivering an active factor, for example, a hormone, to a pre-selected site, for example, the peritoneal cavity, of a mammal. The device comprises a fluid permeable membranous sack for implantation within the mammal and an impermeable hollow tube having one end connected to an opening in the sack and the other end designed to remain outside the body of the mammal. The tube provides an access passageway to the membranous sack, such that after the sack has been surgically implanted into the mammal, a cell containing envelope may be introduced into the sack via the tube. Upon insertion of the cell containing envelope into the sack, the cells may produce an active factor, which subsequently may diffuse into the surrounding tissue or organ of the recipient.

U.S. Pat. No. 4,479,796 describes a surgically implantable dispenser for infusing a pre-selected drug directly into the blood stream. Briefly, the dispenser is surgically spliced in line with a blood vessel. The dispenser encloses a replaceable cartridge of cells, for example, micro-organisms, which produce and secrete the drug into blood flowing past the cartridge. U.S. Pat. Nos. 5,704,910 and 5,911,704 describe an implantable device for delivering a pre-selected molecule, for example, a hormone, into a mammal's systemic circulation. The device comprises an element that is immobilized within a blood vessel and a capsule that is held in place within the blood vessel by the immobilized element. The capsule encloses viable cells which produce and secrete the pre-selected molecule into blood passing the capsule. U.S. Pat. No. 4,309,776 describes an intravascular drug delivery device having a chamber containing transplanted cells for surgical implantation into the wall of a blood vessel. The device comprises a porous wall that permits a hormone, once produced by the transplanted cells, to diffuse out of the chamber and into the blood stream.

Other disorders, however, are caused not by a deficiency, but by the presence and/or excess of a particular molecule in the circulation. For example, a number of autoimmune disorders, for example, myasthenia gravis, Goodpasture syndrome or even type I diabetes, are caused by the abnormal presence of autoantibodies in the systemic circulation. Accordingly, it is possible to treat these disorders by removing the disease causing molecules from circulation via an extracorporeal procedure known as plasmapheresis. Although selective removal is preferable, the development of reliable, cost-effective devices has been lacking (Malchesky et al. (1993) ASAIO J. 39:868-72). In other circumstances, a molecule not normally toxic or harmful to a mammal at normal physiological levels becomes toxic or harmful as its concentration increases. For example, elevated plasma concentrations of β₂-microglobulin in long-term dialysis patients appears to be related to the high frequency of carpal tunnel syndrome and debilitating arthritis in those patients. Similarly, atherosclerosis, a cause of high blood pressure, heart attacks, and strokes, is associated with elevated serum Lipoprotein levels (Ginsberg. (1994) MEDICAL CLINICS OF NORTH AMERICA 78:1-20). Elevated low density lipoprotein (LDL) and very low density lipoproteins (VLDL) level appear to be particularly high risk factors for atherosclerosis.

Furthermore, molecules that ensure the health of an individual can be detrimental if present at elevated concentrations. For example, hormones are essential regulators of body function. However, the same hormones, when present at concentrations higher than those normally found in healthy individuals can lead to a variety of disorders. Similarly, drugs administered to treat a tissue-specific disorder may be harmful to other tissues if their systemic concentrations exceed a critical threshold level.

Although a disorder that is caused by a molecular deficiency can be treated by providing the missing molecule, a disorder that is caused by a molecular excess can be much more difficult to treat. In the case of an excess of β₂-microglobulin, physicians have attempted to treat patients via extracorporeal filtration of the blood to remove the excess β₂-microglobulin. This approach, however, has not been very successful, perhaps because β₂-microglobulin levels quickly return to toxic levels after treatment (Odell et al. (1991) KIDNEY INTERNATIONAL 39: 909-919). Similarly, high plasma LDL levels may be reduced by extracorporeal LDL-apheresis wherein LDL is retained in an adsorbent column (Thompson et al. (1995) LANCET 345: 811-6). This mode of treatment is useful for hypercholesterolaemic patients resistant to drugs, however, its application is limited by its cost and its discontinuous nature, the consequence of which is that low LDL levels post-apheresis rise quickly to near pretreatment levels (Kroon et al. (1999) ATHEROSCLEROSIS 147: 105-113).

Accordingly, it is desirable to produce a device that may be implanted into an animal and that, once implanted, removes a pre-selected molecule from the blood stream. In addition, it is desirable to produce a device that functions continuously over an extended period and may be removed conveniently, if or whenever the necessity arises. Furthermore, it is desirable to provide a method for implanting the device by either non-surgical or minimally invasive surgical procedures.

These and other objects and features of the invention will be more clearly understood from the following description, drawings, and claims.

### SUMMARY OF THE INVENTION

The present invention provides an implantable, intravascular device for ameliorating the symptoms of or preventing a medical disorder associated with the presence and/or concentration of a pre-selected molecule in the systemic circulation of an animal, more preferably a mammal, and most preferably a human. Once implanted, the device removes or causes the removal of the pre-selected molecule from the blood stream over a prolonged period of time. The intravascular device of the invention may be implanted intravascularly using minimally invasive procedures. Furthermore, the intravascular device of the invention is adapted for easy removal using similar minimally invasive procedures to end and/or modify a particular treatment regime. Thus, use of the present device and method provides an easy and reproducible system for removing a potentially harmful molecule from the blood stream of a recipient.

In one aspect, the intravascular device of the invention comprises an anchor adapted for immobilization to an inner wall of a blood vessel, in particular, an inner wall of an intact blood vessel. The anchor is designed such that when immobilized in situ, the anchor permits blood in the vessel to pass therethrough. The device further comprises a cartridge that is retained in place in the blood vessel by the immobilized anchor, which when located in situ also permits blood in the vessel to pass therethrough. The cartridge contains a converting agent, for example, a biocatalyst, for example, a viable cell, or an enzyme, in an amount sufficient to catabolize or modify the pre-selected molecule. During operation, the pre-selected molecule enters into the cartridge where it is catabolized or modified by the converting agent. Accordingly, during operation of the device, the concentration of the pre-selected molecule in the blood stream down stream of the device is lowered relative to the concentration of the pre-selected molecule in the blood stream upstream of the device.

The term “pre-selected molecule” as used herein is understood to mean any substance present in the blood stream, the presence and/or elevated concentration of which is associated with a particular disorder. The pre-selected molecule is associated with a particular disorder when its concentration reaches a level higher than a threshold value found in normal individuals or a population of normal individuals without the disorder. As used herein, the term “elevated concentration” is understood to mean the concentration of a pre-selected molecule that is higher than the concentration normally found in a healthy individual or a population of individuals without the disorder, or is higher than a threshold level above which the disorder is manifested in a particular individual.

Exemplary, pre-selected molecules include, for example, proteins, for example auto-antibodies, hormones and cytokines, lipids, metabolites, drugs, toxins, products of degradative processes, as well as any organic or inorganic molecule, for example, iron, that may accumulate in the blood stream to levels harmful to an individual. The device can be used to catabolize a molecule to treat, ameliorate, prevent, or slow the onset of, a medical disorder associated with the presence of elevated concentrations of the molecule in the blood stream.

In one embodiment, the pre-selected molecule is a protein, for example, β₂-microglobulin or a lipoprotein, for example, LDL and VLDL. It is contemplated, however, that the device may be used to remove from the systemic circulation any molecule that can be catabolized or converted by viable cells or enzymes to one or more molecules that normally are not harmful to the individual, are removed by the recipients own excretory processes, or are not associated with the disorder.

The term “anchor” as used herein is understood to mean any structure immobilizable to an inner wall of a blood vessel, which when immobilized in the blood vessel does not occlude or prevent blood flow through the vessel. The anchor may comprise, for example, at least one element biased in a radially outward direction when immobilized in the lumen of a target blood vessel.

In one embodiment, the anchor may comprise a stent or stent-like element that can be expanded until it becomes radially biased against the inner wall of the blood vessel. Furthermore, the anchor may comprise a barbed or hooked element which can bind the inner wall of the blood vessel. For example, such an anchor may comprise a head and a plurality of barbed or hooked filaments attached to and extending radially from a head such that the filaments are capable of opening umbrella-like until the barbs or hooks located at the end of the filament extending outwardly contact and engage the inner wall of the blood vessel.

In another embodiment, the anchor is an embolism anti-migration filter, such as a blood clot anti-migration filter. A variety of blood clot anti-migration filters, also known as vena cava filters, useful in the practice of the invention are known in the art. A currently preferred anchor is an anti-migration filter known as a “Greenfield® vena cava filter”. Useful Greenfield® vena cava filters are described in U.S. Pat. Nos. 4,817,600 and 5,059,205. Typically, Greenfield® filters comprise a head attached to a plurality of spring biased filaments which, when inserted into the lumen of a blood vessel open, umbrella-like, to contact and grip the inner wall of the blood vessel.

In another embodiment, the anchor may further comprise a receptacle for receiving the cartridge. Moreover, the receptacle may further comprise a locking mechanism to engage and lock the cartridge to the anchor. It is contemplated that both the anchor and the cartridge may comprise interlocking components that mate with one another to lock the cartridge to the anchor.

The term “cartridge” as used herein is understood to mean any structure dimensioned to fit within the lumen of a blood vessel, which when introduced into the blood vessel does not occlude or prevent blood flow through the vessel and having a wall, a least portion of which defines an inner volume that contains the: converting agent for catabolizing or converting the pre-selected molecule.

The term “converting agent” as used herein is understood to mean any agent, for example, a biocatalyst, capable of catabolizing or modifying the pre-selected molecule into one or more molecules that are non-toxic or are less harmful to the host. The term also includes agents that modify the pre-selected molecule to an intermediate which is then removed by a host mediated pathway or response, for example, via an immune response directed against the intermediate or via increased clearance rates by the liver and/or kidney.

In one embodiment, the cartridge comprises at least one hollow fiber. Alternatively, the cartridge may comprise a plurality of hollow fibers, bundled or otherwise associated together. The bundle of hollow fibers may also be retained within a second membrane. The cartridge preferably is designed to facilitate mass transport between the blood stream and the converting agent disposed within the cartridge. In addition to utilizing diffusion as a mechanism for the transport of blood components, the design of the cartridge may also incorporate convective fluxes into and out of the cartridge. Thus, in some circumstances, the flow of blood ultrafiltrate through the cartridge facilitates considerably the transport of reactants (for example, oxygen, nutrients, metabolites and pre-selected molecule) into and products out of the cartridge. In another embodiment, the cartridge further includes a locking mechanism that engages a reciprocal interlocking mechanism on the anchor so that the cartridge can be locked to the anchor in situ.

The hollow fibers preferably are defined at least in part by, for example, a semi-permeable membrane. The semi-permeable membrane defines one or more pores dimensioned to permit entry of the pre-selected molecule into the hollow fiber while at the same time preventing passage of the converting agent out of the hollow fiber into the blood stream. In certain preferred embodiments, for example, in order to provide an immunoprotected environment, the pores permit the passage therethrough of solutes no greater than 150 kD in size. However, under certain circumstances, for example, whenever it is not necessary to provide an immunoprotected environment and convective fluxes are preferred, the pores preferably have a pore size in the range from about 0.1 μm to about 1 μm in diameter. Polymers useful in the manufacture of suitable semi-permeable membranes include, but are not limited to, polyvinylchloride, polyvinylidene fluoride, polyurethane isocyanate, alginate, cellulose and cellulose derivatives (for example, cellulose acetate, cellulose diacetate, cellulose triacetate, cellulose nitrate), polysulfone, polyarylate, polycarbonate, polystyrene, polyurethane, polyvinyl alcohol, polyacrylonitrile, polyamide, polyimide, polymethylmethacrylate, polyethylene oxide, polytetafluoroethylene or copolymers thereof.

In one embodiment, converting agent is a viable cell, for example, a prokaryotic or eukaryotic cell. More preferably, the cell is a eukaryotic cell and most preferably is a mammalian cell, that converts the pre-selected molecule into one or more molecules that are either cleared or capable of being cleared from the circulation of the host. Under certain circumstances, for example, when the cartridge fails to provide an immunoprotected environment, the cells preferably, are autologous cells. In another embodiment, the cells may include a gene that encodes a protein, for example, an enzyme, that once expressed is capable of converting the pre-selected molecule into one or more molecules that preferably are not toxic to the individual. When mass transport into the cartridge is dominated by diffusion and the catalyst comprises viable cells, in order to maintain the viability of the cells, the hollow fibers preferably have an internal diameter of less than about 1000 μm, and more preferably less than about 500 μm.

In another embodiment, the converting agent is an active enzyme. The enzyme can be an isolated enzyme, for example, a partially or substantially pure enzyme preparation. Alternatively, the enzyme can be provided in the form of non-viable cells whereby enzymatic activity is preserved by fixing the cells with a fixative. Furthermore, the enzyme optionally is immobilized on or contained within a solid support, such as a polymeric scaffold, hydrogel, or microcapsule.

It is contemplated that a variety of device configurations may be useful in the practice of the invention. For example, the cartridge may be retained upstream of the anchor, for example, when the cartridge is of a size such that it cannot pass through the anchor. Alternatively, the cartridge may be located downstream of the anchor but retained in place by an attachment means, for example, via a hook or tether, extending from the anchor to the cartridge or via an interlock or fixing mechanism. In addition, it is contemplated that the cartridge and anchor may be configured such that a portion of the cartridge may be located upstream of the anchor with other portions located downstream of the anchor. This type of configuration can be facilitated, for example, via an interlock or fixing mechanism between the anchor and cartridge, or where the cartridge is wedge-like in shape, such that the narrow end of the wedge passes through the anchor but the larger end contacts the anchor thereby to prevent passage of the entire cartridge through the anchor.

In another aspect, the invention provides a method for treating a disorder associated with the presence of a pre-selected molecule in the blood stream of a mammal. The method comprises the steps of (a) introducing into the lumen of a blood vessel a cartridge containing a converting agent capable of catabolizing or modifying the pre-selected molecule; and (b) anchoring the cartridge within the blood vessel.

In one embodiment, the method comprises the additional step of, before introducing the cartridge, immobilizing an anchor to an inner wall of the blood vessel. The anchor, once immobilized can retain the cartridge in situ within the blood vessel. Furthermore, in another embodiment, the method comprises the additional step of locking the cartridge to the anchor.

In this method, the anchor, the cartridge, or both the anchor and cartridge, may be introduced into the blood vessel via a catheter. In one such procedure, the anchor and/or the cartridge may be introduced via catheter into the mammal via a femoral or jugular vein and then immobilized in an artery, arteriole, vein or venule. In a preferred embodiment, the device is immobilized in a natural vein, for example, an inferior vena cava, a superior vena cava, a portal vein or a renal vein, or alternatively, immobilized in a synthetic vein, for example, a vein developed from a surgically-constructed arteriovenous fistula.

### BRIEF DESCRIPTION OF THE DRAWINGS

The present invention will now be more particularly described with reference to and as illustrated in, but in no manner limited to, the accompanying drawings, in which:

FIGS. 1A-D are schematic illustrations of an exemplary implantable, intravascular device located within the lumen of a blood vessel, where the direction of blood flow through the vessel is depicted by an arrow;

FIGS. 2A-C are schematic illustrations showing an exemplary anchor (FIG. 2A), an exemplary cartridge (FIG. 2B), and the exemplary anchor interlocked with the exemplary cartridge (FIG. 2C);

FIGS. 3A-B are schematic illustrations of an exemplary device of the invention (FIG. 3A), and an exemplary device in relation to a device for introducing and/or removing the cartridge (FIG. 3B);

FIGS. 4A-C depict a three-dimensional schematic illustration of an exemplary anchor useful in the practice of the invention (FIG. 4A), a side-sectional schematic illustration of the anchor (FIG. 4B), and a top plan illustration of the anchor (FIG. 4C);

FIGS. 5A-C depict a three-dimensional schematic illustration of an alternative exemplary anchor useful in the practice of the invention (FIG. 5A), a side-sectional illustration of such an anchor (FIG. 5B), and a top plan illustration of such an anchor (FIG. 5C);

FIGS. 6A-D are side-sectional schematic illustrations depicting exemplary pre-filled cartridges useful in the practice of the invention.

FIG. 7 is an exploded cross-sectional illustration of a hollow fiber shown in FIG. 6C;

FIGS. 8A-C are side-sectional schematic illustrations depicting exemplary cartridges useful in the practice of the invention that can be filled and/or refilled in situ;

FIG. 9 is a side-sectional view of another exemplary device useful in the practice of the invention;

FIGS. 10A-D are side-sectional illustrations showing the steps during which an exemplary cartridge is introduced into a blood vessel and engaged via an exemplary anchor immobilized within a blood vessel; and

FIGS. 11A-C are side-sectional schematic illustrations showing the introduction of an empty cartridge into a blood vessel and its filling with converting agent in situ.

In the drawings, like characters in the respective drawings indicate corresponding parts.

### DETAILED DESCRIPTION OF THE INVENTION

In its most general application, the present invention provides an implantable, intravascular device for removing a pre-selected molecule from the systemic circulation of an animal. The device of the invention is adapted for direct implantation into a blood vessel, preferably using a catheter. After implantation, the device permits the pre-selected molecule to pass from the blood stream of the host into the device where it is catabolized or converted into one or more molecules that are less harmful to the individual than the pre-selected molecule and/or are not associated with the disorder.

The intravascular device of the invention potentially can be used to treat a variety of disorders which result from the accumulation of a variety of molecules within the circulation. Elevated levels of certain circulating molecules may cause hematologic, metabolic, endocrinologic, neurologic, hepatic, renal, and immunologic disorders. The origin of such molecules may arise from any or more of the above systems. Examples of such disorders include, for example, dialysis-related amyloidosis caused by an excess of circulating β₂-microglobulin; hemochromatosis caused by an excess of iron; severe combined immunodeficiency (SCID), one form of which results in an excess of adenosine; endocrine disorders including congenital adrenal hyperplasia which cause an accumulation of certain steroid precursors; familial defective APO-B100 which causes elevated plasma cholesterol levels and accelerated atherogenesis; lipoprotein lipase deficiency, which causes elevated levels of triacylglycerol and low-density lipoproteins and results in atherosclerotic vascular disease; Crigler-Najjar, Dubin-Johnson, and Rotor's Syndromes which result in high levels of bilirubin and irreversible neurologic damage in infants. Inborn Errors of Metabolism may also cause an accumulation of toxic levels of a molecule in the bloodstream. Examples include Lesch-Nyhan Syndrome which causes an overproduction of purine nucleotides and accumulation of 5-phosphoribosyl-1-pyrophosphate and uric acid; the aminoacidurias, including phenylketonuria, which causes an accumulation of phenylalanine, tyrosinemia, which is an excess of tyrosine, alkaptonuria, which results in toxic accumulations of homogentisate, the Branched-Chain Amino Acidurias, such as maple syrup urine disease (MSUD), a toxic accumulation of branched-chain amino acids, homocystinuria, an excess of homocysteine, and hyperoxaluria, an excess of oxalate. Other examples of inborn errors of metabolism include galactosemia, which results in toxic accumulation of galactose and galactose-1-phosphate in tissues; citrullinemia, an excess of citrulline.

Dialysis-related amyloidosis results from an accumulation in the blood stream of β₂-microglobulin (β₂M) because of insufficient removal by failing kidneys. Thus, removal of β₂M from the bloodstream may slow down the progression and/or alleviate the symptoms of amyloidosis. Removal of β₂M from the bloodstream may be achieved, for example, by incorporating into the device of the invention proximal tubule cells which take up and metabolize β₂M. Accumulation of unconjugated bilirubin, caused by errors in conjugation, uptake or excretion, leads to Crigler-Najjar, Rotor's syndrome, Dubin-Johnson syndrome, or Gilbert's syndrome. Accordingly, the use of a device containing the requisite enzymatic activity, such as glucuronosyltransferase, has the potential to cure and/or alleviate the symptoms of these disorders.

The intravascular device of the invention comprises an anchor and a cartridge. The anchor is dimensioned for insertion into the lumen of an intact blood vessel. Once introduced to a desired location in vivo, the anchor is immobilized to an inner wall of the blood vessel. The anchor is designed such that when immobilized to the wall of the blood vessel, the element permits blood in the vessel to pass therethrough. The cartridge likewise is dimensioned for insertion into the lumen of the blood vessel. The cartridge is retained in situ by virtue of the anchor. The cartridge contains a converting agent, for example, a biocatalyst, for example, a viable cell and/or an enzyme preparation that catabolizes or otherwise modifies the pre-selected molecule. During operation, the device removes or induces the removal of the pre-selected molecule from circulation such that the concentration of the pre-selected molecule in the blood stream downstream of the device is lower than the concentration of the pre-selected molecule upstream of the device. Proper operation of the blood conditioning device of the invention requires, therefore, that it not occlude the blood vessel, i.e., the device does not prevent passage of blood through the blood vessel.

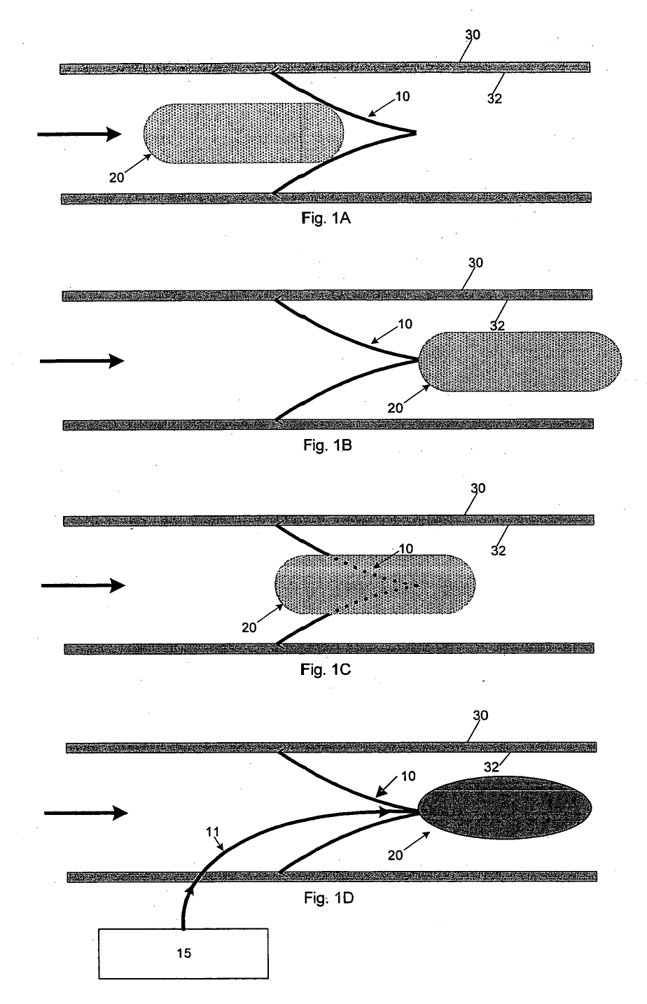

The device of the invention is described in more detail with reference to the drawings, which are provided for purposes of illustration and are not meant to be limiting in any way. FIG. 1 shows side view illustrations of exemplary configurations of implantable devices of the invention. In FIG. 1, the arrows represent the direction of blood flow. FIG. 1A depicts anchor 10 and cartridge 20, wherein anchor 10 is immobilized in blood vessel 30, more specifically to an inner wall 32 of intact blood vessel 30. The cartridge 20 is located upstream of the immobilized anchor 10. In FIG. 1B, cartridge 20 is located downstream of anchor 10 immobilized to an inner wall 32 of an intact blood vessel 30. In FIG. 1C, the cartridge 20 is positioned relative to anchor 10 immobilized to an inner wall 32 of a blood vessel such that a portion of cartridge 20 is located upstream of anchor 10 and a portion of cartridge 20 is located downstream of anchor 10. In FIG. 1D (which is similar to FIG. 1B), cartridge 20 is located downstream of anchor 10 immobilized to an inner wall 32 of intact blood vessel 30. The device has been modified to include a conduit 11 connecting cartridge 20 to an extravascular element 15 (for example, a reservoir, a pump, and/or a vascular access port) containing converting agent, so that as the converting agent in cartridge 20 is used up, cartridge 20 can be refilled or recharged with converting agent from extravascular element 15. It is contemplated that the devices shown in FIGS. 1A and 1C may also be modified as such to permit filling of the cartridge in situ. For example, an extravascular element also can be used in combination with an intravascular reservoir located with respect to the anchor as shown in FIGS. 1A and 1C. Additional designs and design considerations can be found in copending U.S. patent application Ser. No. ______, filed on even date herewith, entitled “Intravascular Drug Delivery Device and Use Thereof,” and assigned attorney docket number NPH-003, which claims priority to and the benefit of U.S. Ser. No. 60/250,746. The entirety of each of these applications is incorporated herein by reference.

The mechanism by which cartridge 20 is retained by anchor 10 may vary depending upon the relative configuration of the components of the device. For example, in the configurations shown in FIGS. 1A and 1C, cartridge 20 can be retained in position by contacting anchor 10 where cartridge 20 is dimensioned such that it is too large to pass entirely through the anchor 10. However, it is contemplated that in the configurations shown in FIGS. 1A-1C, cartridge 20 may be locked or otherwise physically tethered to anchor 10 via a locking or tethering mechanism.

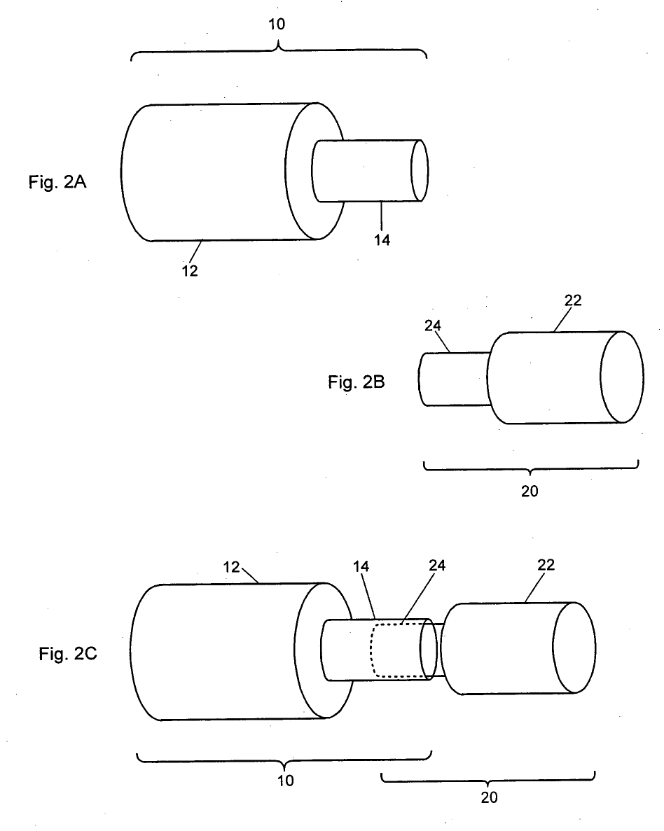

FIGS. 2A-2C are schematic illustrations of an exemplary two component system and depict anchor 10 (FIG. 2A), cartridge 20 (FIG. 2B), and an exemplary blood conditioning device in which the components are locked together (FIG. 2C). In FIG. 2A, anchor 10 comprises a first element 12, connected to a second element 14. First element 12 is adapted for radial interference fit with the inner wall of an intact blood vessel. Second element 14 forms a receptacle for mating with a reciprocal locking member of cartridge 20. In FIG. 2B, cartridge 20 comprises a first element 24 connected to a second element 22. The first element 24 defines a locking member that engages a reciprocal locking member of the anchor 10. The second element 22 contains a wall, at least a portion of which defines an inner volume for retaining the converting agent. In FIG. 2C, the anchor 10 is locked to cartridge 20. The second element of the anchor 14 engages and locks the first element of cartridge 24.

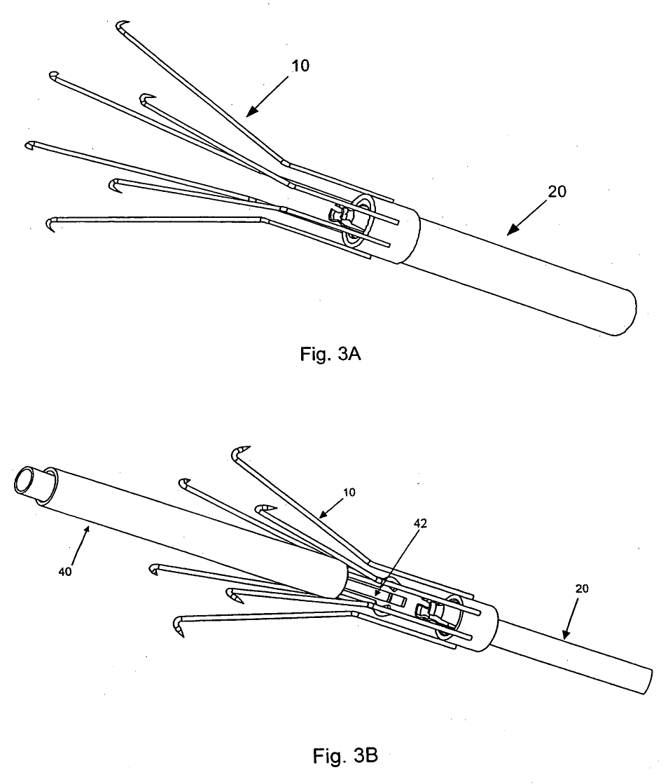

FIG. 3A is a three-dimensional illustration of the device of the invention. In FIG. 3A, anchor 10 is shown engaged to cartridge 20. In FIG. 3B an introduction catheter 40 and a grabbing device 42 disposed within catheter 40 are shown in relation to interlocked anchor 10 and cartridge 20.

Upon implantation, the cartridge is held securely in place via the anchor. A cartridge of appropriate design can be introduced into the bloodstream upstream of the anchor which is then transported downstream by blood flow until it is captured passively by the preimplanted anchor, irrespective of the presence or absence of an appropriate locking mechanism between anchor and cartridge. In a preferred embodiment, however, the anchor and cartridge have interconnecting locking mechanisms so that the cartridge can be locked securely in place with the anchor. The incorporation of a locking mechanism can obviate the requirement of introducing the cartridge upstream of the anchor. Thus, use of a locking mechanism enables the implantation of heavier cartridges for which gravitational forces are significant in comparison to the applied hydrodynamic force. The locking mechanism preferably is designed to permit the capture and engagement of the cartridge and, if required, to permit the release of the cartridge.

There are a number of ways to removably attach the cartridge to the anchor, in situ, via a mechanical fastener methods, either with or without an interference fit. For example, an outer wall portion of the cartridge can be sized to provide a radial interference fit with a bore or collar in the anchor formed by compliant resilient members, such as cantilevered beams, expandable mesh strands, one or more spring loaded devices or levers, and the like. Alternatively or additionally, the device may comprise a positive mechanical interlock with mating male and female portions, as are known to those skilled in the art of mechanical fastening. Examples include, but are not limited to, threaded members, bayonet retention fittings, ratchet tooth locking latch clamps, and the like. Attachment and/or removal of the cartridge may be accomplished by rotation, translation, or a combination of rotation and translation. Additionally, a catheter can employ an end effector configured to actuate a structure on the cartridge and/or the anchor to facilitate attachment and/or removal, for example, by temporarily expanding a bore, constricting a wall, displacing a latch, opening or closing a clamp, and crimping a compliant member.

The intravascular device of the invention is capable of catabolizing or modifying the pre-selected molecule over a prolonged period of time, preferably in range of weeks, for example, one, two, three or four weeks, and more preferably in the range of months, for example, two, three, four, five, six, seven, eight, nine, ten, eleven or twelve months. It is contemplated, however, that exhausted cartridges, for example, wherein a substantial fraction of the converting agent disposed within the cartridge is no longer able to catabolize or modify the pre-selected molecule, may be retrieved from the recipient and replaced with new cartridge containing new or even different converting agents to restore or modify the treatment protocol.

The Anchor

The art is replete with anchors useful in the practice of the invention. Useful anchors are characterized by their ability to be immobilized within the lumen of a blood vessel without occluding or preventing blood flow through the blood vessel, while still providing, as is or after modification, a secure and flexible way to retain the cartridge.

Commercially available embolism anti-migration filters and stents are exemplary anchors which lack locking mechanisms that are useful in the practice of the invention. Stents typically are used routinely by medical practitioners to increase the internal diameter of blood vessels to restore or maintain patency. Blood clot anti-migration or vena cava filters also are used routinely by medical practitioners but are used to prevent the migration of potentially life threatening blood clots within the vasculature. Blood clot anti-migration filters typically are designed to be implanted and anchored within the lumen of a blood vessel. When implanted, the anti-migration filters permit blood in the vessel to pass by while simultaneously trapping blood clots. Anchors may be obtained commercially and used as is, or more preferably adapted to further include a locking mechanism that can engage a reciprocal locking member on the cartridge.

The art is replete with helical, cylindrical and/or tubular stent designs capable of modification for use in the instant invention. For example, the stents disclosed in U.S. Pat. Nos. 5,370,691, 5,591,230, 5,651,174, 5,899,935, 5,895,407, 6,107,362, 6,207,516, 6,030,414 and 6,036,725 may be modified to receive and/or engage a cartridge. Furthermore, a variety of percutaneous catheter and guidewire systems may be used to introduce and deploy at a desired location stents useful in the practice of the invention (see, for example, U.S. Pat. Nos. 5,891,154 and 6,027,520).

A variety of blood clot anti-migration filters useful in the practice of the invention are known in the art and are available commercially. For example, blood clot anti-migration filters like those described in U.S. Pat. Nos. 4,817,600 and 5,059,205, are available from Medi.Tech®, Boston Scientific Corporation, Mass., and are particularly well suited for use as an anchor element. In particular, these filters are designed to provide maximal entrapment area for trapping blood clots while maintaining patency of the blood vessel after trapping emboli. For example, the geometry of the cone-shaped filters permits filling to 80% of its depth before the cross-sectional area is reduced by 64%, and that at least 80% of the depth of the filter can be filled without development of a significant pressure gradient across the filter. The spacing between the six legs of these filters ensures the trapping of emboli greater than 3 mm (Greenfield et al. (1989) “Venous Interruption” Chapter 68, pp. 929-939 in HAIMOVICI'S VASCULAR SURGERY PRINCIPLES AND TECHNIQUES THIRD EDITION, Appleton and Lange, Norwalk, Conn./San Mateos, Calif.). Accordingly, the filters may be used as such to capture a cartridge greater than 3 mm in diameter. Other blood clot anti-migration filters useful, either as is or after modification by inclusion of an interlocking mechanism, in the practice of the invention are described, for example, in U.S. Pat. Nos. 4,494,531, 4,781,177, 4,494,531, 4,793,348, 4,832,055, 5,152,777, 5,350,398, 5,383,887, 5,720,764, 6,059,825, 6,080,178, and 6,126,673. Also, it is contemplated that other blood clot anti-migration filters, such as those described in Greenfield (1991) in VASCULAR SURGERY, A COMPREHENSIVE REVIEW, Moore, ed. W.B. Saunders Co., Philadelphia, London, Toronto, Montreal, Sydney, Tokyo pp. 669-679, including, for example, Nitinol filters; Gunther filters; Venatech filters; Amplatz filters; and birds nest filters, likewise may be used in the practice of the invention.

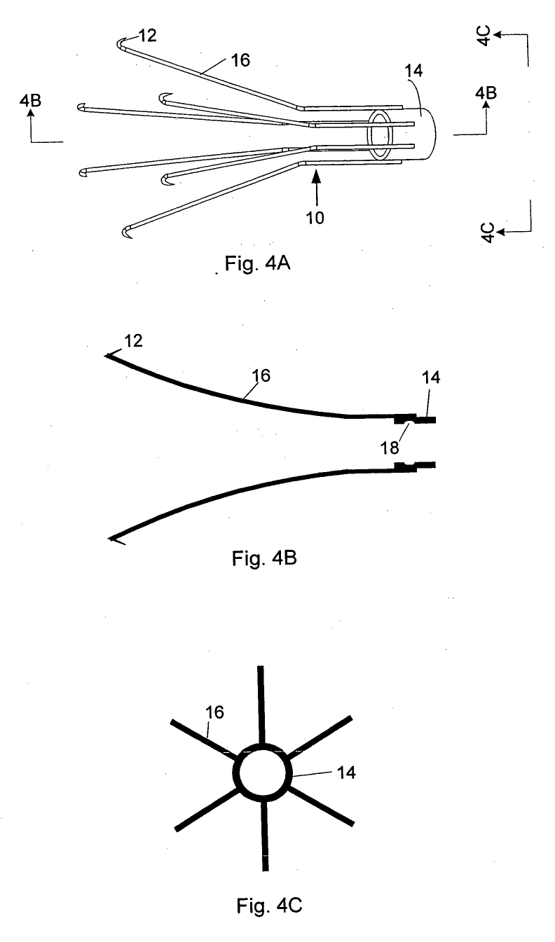

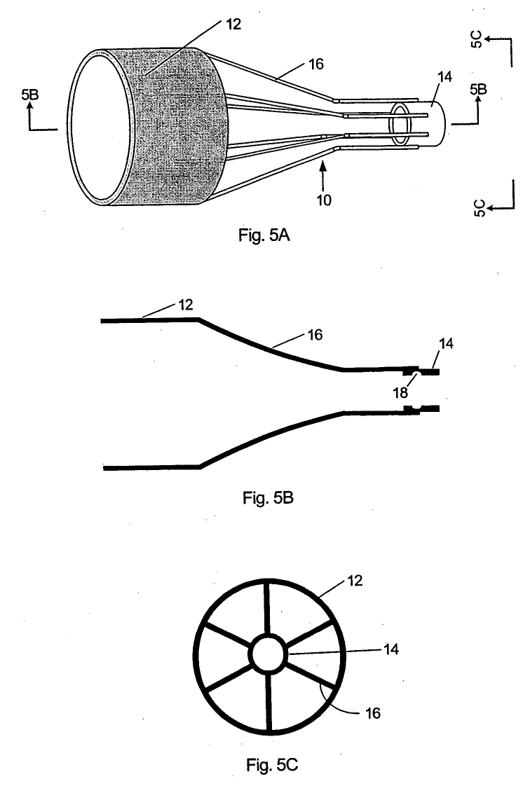

Although commercially available anti-migration filters can be used in the device of the invention, it is preferable that the anchor incorporates a locking mechanism to engage the cartridge (see, FIG. 4). Commercially available stents typically do not possess a means for capturing a cartridge. However, such stents can be modified, for example, by incorporating an extension comprising legs and a receiving member as shown in FIG. 5. Alternatively, stents can be used as such if, for example, the cartridge comprises legs with appropriate hooks or barbs that engage a blood contacting surface of the stent. In the latter case, the primary role of the stent is to spread the force applied by the hooks/barbs to a wide surface area and thus minimize the risk of cartridge migration and to provide the means for repeated implantation/retrieval of the cartridge, while avoiding injury to the vessel wall.

It is preferable, however, that new anchors incorporating locking heads, such as the anchor elements shown in FIGS. 4 and 5, are designed and manufactured to better fit the requirements of the present invention. The anchor element may be synthetic or metallic. Preferably, at least a portion of the anchor is metallic and more preferably at least a portion of the anchor is made from titanium due to its light weight, strength and biocompatibility.

Two preferred anchors useful in the practice of the invention are presented in FIGS. 4 and 5. In particular, FIG. 4 shows in more detail the anchor element shown in FIG. 3. In FIG. 4A, anchor 10 comprises a head 14 and a plurality of resilient, typically metallic legs 16 extending therefrom. The end of the legs distal to the head comprise hooks or barbs 12 disposed outwardly to engage an inner wall of the;target blood vessel. FIG. 4B shows in cross section, head 14 incorporating a locking mechanism 18 which, as described in detail below, is used to engage a reciprocal locking mechanism of the cartridge. FIG. 4C shows in top plan view legs 16 extending radially from head 14. The hooks or barbs 12 of FIG. 4A correspond to first element 12 of FIG. 2A, and head 14 of FIG. 4A corresponds to the second element of FIG. 2A. Leg 16 in FIG. 4A corresponds to a third element that connects the first element (hook or barb) 12 to the second element (head) 14.

An alternative anchor design is shown in FIG. 5. In FIG. 5A, the anchor comprises a head 14 and a plurality of legs 16 extending from head 14 at one end to a stent 12 at the other end. Stent 12 can be a self-expandable stent or can be deployed with the aid of a balloon, or can be any other stent design known in the art. FIG. 5B is a cross-sectional view of the anchor shown in FIG. 5B and shows the spatial relationship of stent 12, legs 16 and head 14, as well as a locking mechanism 18 incorporated in head 14. As described below, the locking mechanism engages a reciprocal locking mechanism of the cartridge. FIG. 5C is a top plan view of the anchor shown in FIG. 5A and shows the spatial relationship between head 14, legs 16 and stent 12.

The primary difference between the anchors shown in FIGS. 4 and 5 is the way in which each anchor is adapted to contact and engage the inner wall of a blood vessel. In the anchor shown in FIG. 4, the outwardly extending barbs may be preferable for implantation inside a vein. This system takes advantage of the relatively low venous blood pressure to minimize the contact area and thus possible negative interaction between vessel and implant. On the other hand, in the anchor shown in FIG. 5, a stent may be preferable for implantation inside an artery, i.e., a high pressure blood vessel. This system takes advantage of the large contact area between the stent and blood vessel ensuring that hydrodynamic and gravitational forces applied to the implant are spread over a large surface area, thereby minimizing the potential for arterial wall injury or anchor migration.

Cartridge and Cartridge Design

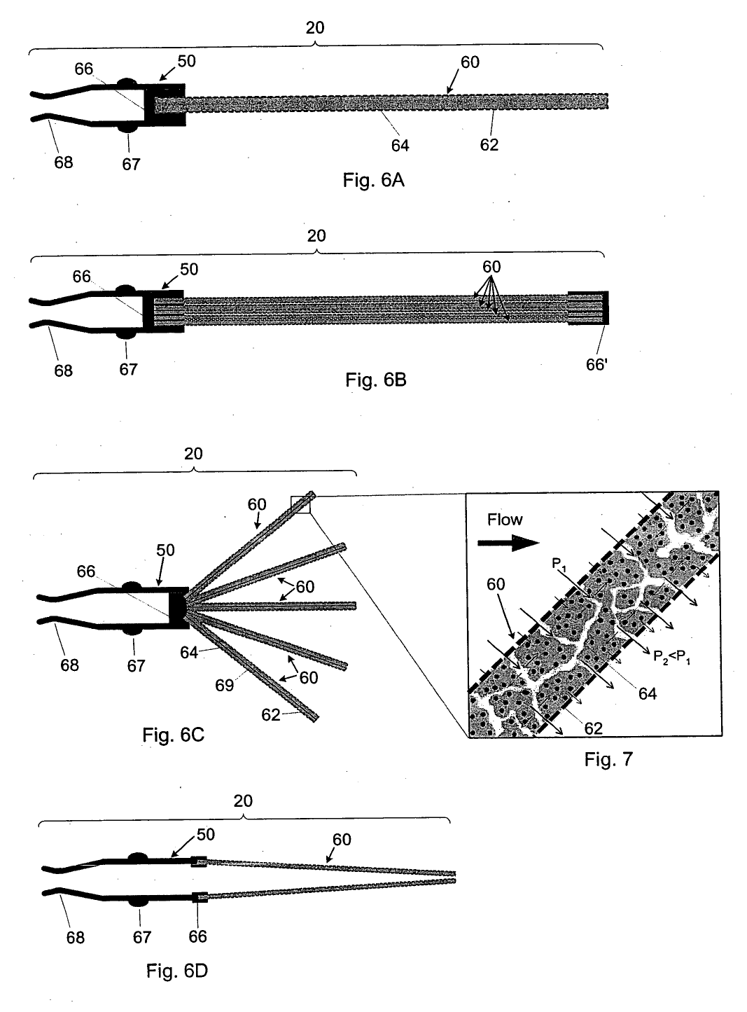

It is contemplated that a variety of cartridge designs may be useful in the practice of the invention. Exemplary cartridges are shown in FIGS. 6-9 and are discussed in more detail below. Optimal design, however, will depend upon a variety of considerations, including, for example, the source and nature of the converting agent, mass transfer characteristics and requirements, and hemocompatibility. For example, when a small amount of the converting agent is required to remove a pre-selected molecule from the bloodstream, the converting agent may be incorporated in a single hollow fiber, for example, as illustrated in FIG. 6A. Other applications requiring higher amounts of the converting agent may require that a number of hollow fibers be combined to increase available volume; the fibers may be held together in a bunch (FIG. 6B) or diverge to permit blood to flow between them (FIG. 6C). FIGS. 6A and 6B represent cartridges in which mass transport of solutes into and out of the hollow fibers are governed primarily by diffusion. In contrast, FIGS. 6C and 6D represent cartridges in which mass transport into and out of the hollow fibers occurs by convection in addition to diffusion. The exemplary cartridges are described in more detail below.

FIG. 6A illustrates an exemplary cartridge 20 comprising a single hollow fiber 60 attached to collar 50. The hollow fiber 60 is defined by a membrane 62 (for example, a semi-permeable membrane) at least a portion of which defines a cavity or inner volume 64 for containing the converting agent. Collar 50 includes an end cap 66 for attaching hollow fiber 60 to collar 50. Collar 50 also includes an interlocking mechanism 67 capable of engaging a reciprocal interlocking mechanism of the anchor. Furthermore, collar 50 is adapted to include a seizable element 68, that can be seized by a grabber element to facilitate introduction of the cartridge into a recipient and/or removal of the cartridge from the recipient.

FIG. 6B illustrates an exemplary cartridge 20 similar to that shown in FIG. 6A, except that it has a higher capacity for holding greater amounts of converting agent. The major difference is that cartridge 20 comprises a plurality of hollow fibers 60 which are bundled together. Although, FIG. 6B shows the hollow fibers 60 bundled together by means of end caps 66 and 66′, of which end cap 66 is associated with collar 50, it is appreciated that the hollow fibers may be bundled together by means of a single end cap 66. In the latter example, the hollow fibers when placed in situ will be free to move around relative to one another. As in FIG. 6A, collar 50 also includes an interlocking mechanism 67 capable of engaging a reciprocal interlocking mechanism of the anchor. Furthermore, collar 50 is adapted to include a seizable element 68, that can be seized by a grabber element to facilitate introduction of the cartridge into a recipient and/or removal of the cartridge from the recipient.

FIG. 6C illustrates an additional exemplary cartridge 20 in which a plurality of hollow fibers 60 are attached or bundled together by one of their ends via end cap 66 disposed in collar 50. In this embodiment, the hollow fibers each contain a spring loaded filament 69 which open up umbrella-like once the cartridge is located in situ. As a result, when implanted, the hollow fibers are located at an angle relative the flow of blood through the blood vessel. Collar 50 also includes an interlocking mechanism 67 capable of engaging a reciprocal interlocking mechanism of the anchor. Furthermore, collar 50 is adapted to include a seizable element 68.

FIG. 6D illustrates another exemplary cartridge 20. In FIG. 6D, a single tubular hollow fiber 60 is attached to collar 50 via an annular end cap 66. Both ends of the hollow fiber 60 are open to permit blood flow into and out of the tube. The converting agent is disposed inside the wall of the tubular hollow fiber. The tubular hollow fiber is dimensioned such that the internal diameter of the hollow fiber tube closest to collar 50 is larger than the internal diameter of the tube at the end opposite from that attached to collar 50. Continuous flow through the wedge-shaped tube formed by the hollow fiber prevents flow stagnation and blood clot formation while it increases local blood pressure to levels higher than those in the blood passing around but not through the tube. As a result, convective fluxes in the direction from the inside to the outside of the tube through hollow fiber 60 transports materials into and out of the internal space of hollow fiber 60. Collar 50 also includes an interlocking mechanism 67 capable of engaging a reciprocal interlocking mechanism of the anchor. Furthermore, collar 50 is adapted to include a seizable element 68.

FIG. 7 is an exploded cross-sectional view of a single angled hollow fiber as illustrated in FIG. 6C. In this design, most of the blood is directed around the hollow fibers, however, a portion of the blood is transported through the fiber. As a result of blood flow, the pressure P₁ at the side of the fiber proximal to collar 50 is larger than the pressure P₂ at distal side of the fiber. The rate of mass transport is determined by the pressure gradient P₁-P₂ as well as the transport properties, such as hydraulic permeability and the molecular weight cut-off of the semi-permeable membrane, and the converting agent formulation in the hollow fiber. Because hydraulic permeability is higher for large pores, most convective flow occurs through such pores. Diffusion also takes place, both through the wall of the hollow fiber membrane and in the interior of the hollow fiber. This combination of convection and diffusion can be utilized in preferred embodiments to enhance mass transport to levels far higher than those achievable in extravascular implants. As a result, a device designed to incorporate convective transport may support larger hollow fiber dimensions and greater densities of converting agent (for example, viable cell) than those having mass transport governed solely by diffusion.

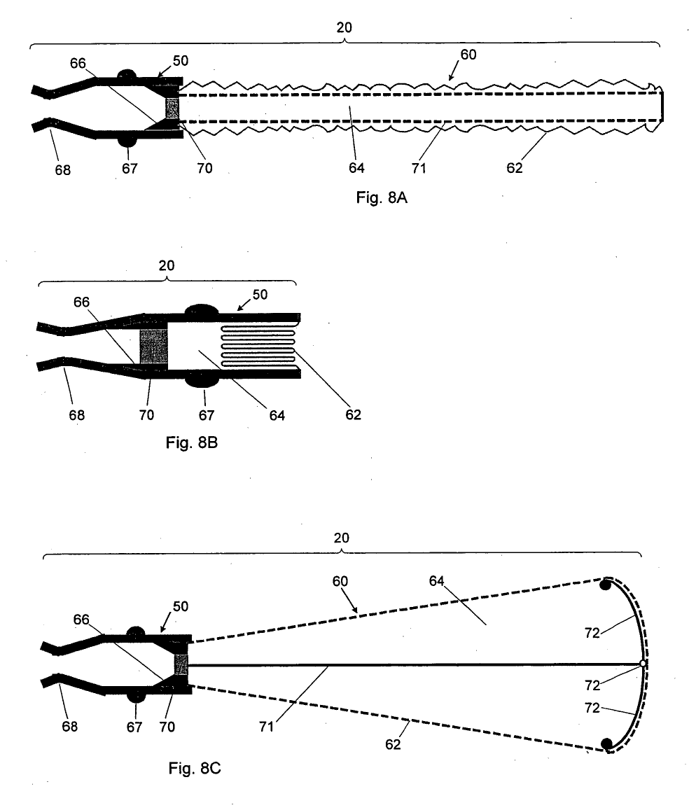

FIGS. 8A-8C represent exemplary cartridges that can be loaded with converting agent once immobilized in situ. In the cartridges shown in FIGS. 8A and 8B, mass transport into and out of the hollow fiber may occur primarily by diffusion. In contrast, in the cartridge shown in FIG. 8C, mass transport into and out of the hollow fiber occurs by both convention and diffusion.

FIG. 8A illustrates a cartridge 20 comprising collar 50 and hollow fiber 60. Hollow fiber 60 is defined by a flexible permeable membrane 62 built around a solid supporting frame 71, for example a perforated tubular frame, to define inner volume 64. The length of the cartridge is fixed whether empty or loaded while its diameter is substantially that of supporting frame 71 when empty but, like a balloon, its diameter increases to that defined by the surface area and elasticity of the flexible membrane when loaded. Hollow fiber 60 is attached to collar 50 by end cap 66. The cartridge further comprises a septum 70 which seals the inner volume 64 of the cartridge but yet permits drug to be loaded into the cartridge once located in situ. Collar 50 also includes an interlocking mechanism 67 capable of engaging a reciprocal interlocking mechanism of the anchor. Furthermore, collar 50 is adapted to include a seizable element 68.

FIG. 8B illustrates a second exemplary, empty cartridge but lacking a solid support frame. In this type of cartridge, membrane 62 of the empty cartridge 20 is folded inside the cavity defined by collar 50 and is released from the cavity outwardly due to the positive pressure generated during the in situ loading of interior volume 64. The membrane material and dimensions must in this case be selected such that upon loading the membrane, like a balloon, assumes the desired elongated rather than spherical shape and maintains the required strength. The cartridge further comprises a septum 70 which seals the inner volume 64 of the cartridge but yet permits drug to be loaded into the cartridge once located in situ. Collar 50 also includes an interlocking mechanism 67 capable of engaging a reciprocal interlocking mechanism of the anchor. Furthermore, collar 50 is adapted to include a seizable element 68.

FIG. 8C illustrates another exemplary cartridge 20 comprising collar 50 and hollow fiber 60. Hollow fiber 60 is attached to collar 50 via end cap 66. Hollow fiber 60 is defined by a permeable membrane 62 built around a supporting frame 71. One end of frame 71 is attached to collar 50. The other end of hollow frame 71 is attached to outwardly extending filaments 72 via hinge 73. During implantation, filaments 72 are bent towards supporting frame 71, however, can move away from frame 71 umbrella-like during implantation thereby generating a wedge-shaped hollow fiber. Such a shape facilitates the creation of pressure gradients that induce convective fluxes. The expansion of filaments 72 may be automatic (for example, via spring loaded filaments) after cartridge deployment from the catheter, or may be performed manually by appropriate operator action via a catheter system (for example, by pushing or pulling a wire extending from the cartridge). The cartridge further comprises a septum 70 that permits drug to be loaded into the cartridge once located in situ. Collar 50 also includes an interlocking mechanism 67 capable of engaging a reciprocal interlocking mechanism of the anchor. Furthermore, collar 50 is adapted to include a seizable element 68. With this type of cartridge, blood flows around the wedge-shaped fiber from the narrower to the wider portion. Blood pressure rises along the length of the hollow fiber and then drops after the blood passes the end of the hollow fiber. When combined with highly permeable membranes, the pressure gradients can lead to substantial convective currents that enhance mass transfer.

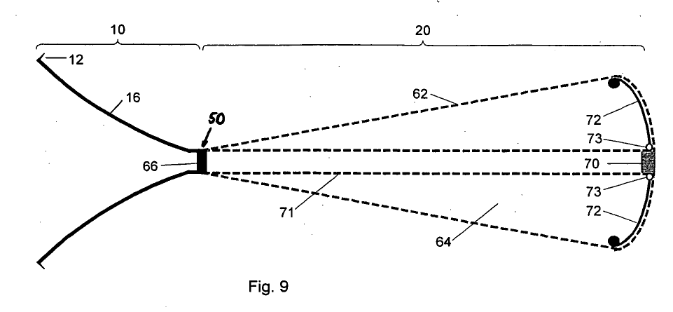

FIG. 9 illustrates another exemplary device useful in the practice of the invention and includes an integrated anchor 10 and cartridge 20. The cartridge can be loaded with converting agent in situ. Anchor 10 comprises hooks or barbs 12 attached to one end of filament 16. The other end of filament 16 is attached to collar 50. This type of configuration obviates the need for a separate anchor. Semi-permeable membrane 62 (attached to collar 50) is built around a solid supporting frame 71, for example a perforated tubular frame. Spring loaded filaments 72 are attached to supporting frame 71 via hinges 73. During implantation via a catheter, filaments 72 are collapsed around frame 71. Once at the appropriate location, leg filaments 16 open to engage the inner wall of the blood vessel or the blood contacting surface of a pre-implanted stent, and spring loaded filaments 72 open to define the shape of the cartridge. The shape of the cartridge induce blood flow characteristics that support convective flow through the cartridge. In this design, septum 70 is located on a side of the cartridge opposite to that attached to collar 50 and end cap 66 is attached to collar 50. However it is contemplated that the relative positions of septum 70 and end cap 66 can be reversed.

A variety of cartridges having different shapes may be useful in the practice of the invention. A preferred cartridge shape is described in detail in Example 2. The preferred shape is designed to minimize turbulence in the blood passing the implanted cartridge. The shape of the upstream end of the cartridge appears to be less critical than the shape of the downstream end of the cartridge. In particular, the downstream end of the cartridge preferably is tapered to an apex so as to minimize a wake effect. A variety of shapes for the upstream end of the cartridge may be used, however, under certain circumstances, it may be advantageous to use a flow directing member to direct the flow of blood around the cartridge. The flow directing member may be conical in shape with the apex of the member located upstream and the base of the member located downstream relative to the cartridge.

The appropriate inner volume for the cartridge depends upon a variety of considerations. One consideration, for example, includes the biological activity of the converting agent, for example, the productivity of cells or the activity of the enzyme, to be incorporated into the device. For example, if a first type of converting agent removes a pre-selected molecule more efficiently than a second type, then less converting agent of the first type will be needed to remove the same amount of pre-selected molecule. Other considerations include, for example: the amount of catabolism/removal necessary to produce the desired therapeutic effect in the recipient; rates of mass transport from the blood into the cartridge and vice versa to provide the pre-selected molecule and reactants to the converting agent and to dissipate the products of the converting agent back into the blood; the time over which the converting agent must remain active post implantation, and the density to which the converting agent can be incorporated to optimize its functionality and longevity. Once these variables have been established, then by judicious choice of cartridge geometry and converting agent it is possible to create a cartridge capable of removing the desired amount of pre-selected molecule for the longest period of time desired.

Because the conversion rate depends both on the amount of the converting agent and certain other components, for example, the amount of the pre-selected molecule as well as other secondary agents necessary for the converting agent to work, it is important that the cartridge be designed to facilitate mass transport between the bloodstream and the inner volume of the cartridge. While mass transport considerations are very important for the efficient function of enzymatic activity, they are even more critical for living cells because they may result in loss of cellular viability and consequently of catalytic activity, for example, due to limitations in oxygen transport. In the case of enzyme preparations, the cartridge preferably is dimensioned to optimize the transport of reactants between blood and the enzyme. In the case of viable cells, the cells require the supply of a variety of nutrients and oxygen, and the removal of cellular byproducts to maintain cell viability. Accordingly, the design must ensure adequate transport rates for all critical molecules. However, oxygen transport is the most important aspect in maintaining cell viability and, therefore, in most embodiments incorporating viable cells, oxygen transport is a limiting parameter.

Because the rate of diffusion-based mass transport diminishes very rapidly as the size of the diffusing molecule increases, its concentration decreases, and/or diffusion distance increases, it is preferable to use a cartridge that supports convective mass transfer between blood and the converting agent. Examples of preferred cartridge designs that induce convective transport are shown in FIGS. 6C, 6D, 8C and 9. All of these designs introduce pressure gradients across the permeable cartridge containing converting agent.

In some embodiments, however, the incorporation of convective transport may not be necessary to ensure long-term functionality of the cartridge. For example, diffusion alone may be adequate in applications where a small amount of converting agent is needed to remove a pre-selected molecule of low molecular weight (i.e., has high diffusivity). Even in these cases, the cartridge must be designed to ensure adequate diffusion rates for the limiting reactants, for example, oxygen in the cases of viable cells. In the case of oxygen, studies have shown that, in order to maintain the viability of cells excluded from the blood stream or a blood supply, the cells preferably are located within a critical diffusion distance of about 500 μm, more preferably within about 300 μm of the blood supply.

The hollow fibers preferably are produced from a semi-permeable membrane having pores dimensioned to permit the diffusion of the pre-selected molecule into the lumen of the hollow fiber while permitting the efflux of waste products out of the hollow fiber. In addition, the pores preferably are dimensioned to exclude the passage of converting agent therethrough. Accordingly, the pores are designed to prevent migration of converting agent from the lumen of the hollow fiber into the blood steam, thereby maintaining the converting agent at a single location in the host to facilitate subsequent removal, if or when necessary.

In addition, under certain circumstances, it is contemplated that, although the pores should be large enough to permit the entry of the pre-selected molecule, the pores preferably should exclude molecules, for example, antibodies and cytotoxic blood components, having a molecular weight greater than about 150 kD. Furthermore, under certain circumstances the pores may also be designed to prevent the influx of the host's immune cells, for example, macrophages and lymphocytes, which if allowed to enter the lumen of the hollow fibers may be detrimental to the activity of the converting agent disposed therein. The membrane, upon choice of an appropriate pore size, can provide an immunoprivileged or immunoprotected environment that protects the cells or enzymes enclosed therein from an immune response. This may be an important consideration if the implanted converting agent, for example, the viable cells are non-autologous in nature.

It is appreciated that, if the pre-selected molecule has a molecular weight exceeding 150 kilodaltons, then cells disposed within the hollow fiber preferably are autogeneic or autologous in nature. It is contemplated that, the autogeneic or autologous cells elicit a weaker immune response than cells from other sources, and as a result have enhanced viability and longevity. However, if the pre-selected molecule has a molecular weight less than 150 kilo daltons, then it is anticipated that any cell type may be entrapped with the hollow fiber, although autogeneic or autologous cells are preferred.

The cartridge and/or the hollow fibers may be produced from biocompatible polymers which include, but are not limited to, polyvinylchloride, polyvinylidene fluoride, polyurethane isocyanate, polyalginate, cellulose or cellulose derivatives (cellulose acetate, cellulose diacetate, cellulose triacetate, cellulose nitrate), polysulfone, polystyrene, polyurethane, polyvinyl alcohol, polyacrylonitrile, polyamide, polyimide, polymethylmethacrylate, polyethylene oxide, polytetrafluoroethylene or copolymers thereof. A summary of currently available hollow fibers, including methods of manufacture and the names of commercial suppliers, is set forth in Radovich (1995) “Dialysis Membranes: Structure and Predictions,” Contrib Nephrol., Basel, Karger, 113: 11-24. In addition, useful polytetrafluorethylene polymer hollow fibers are available commercially from Impra, Inc., Tempe, Ariz. or W.L. Gore and Associates, Flagstaff, Ariz.

As discussed previously, if enough converting agent can be enclosed within a single hollow fiber to catabolize a sufficient amount of the pre-selected molecule in the blood stream, then the cartridge of the invention preferably contains a single hollow fiber. Alternatively, if the requisite amount of converting agent cannot be enclosed within a single hollow fiber then the converting agent may be entrapped within a bundle of hollow fibers, wherein bundle of fibers optionally are further encapsulated within a second macroporous outer membrane. The porous outer membrane preferably defines pores that do not affect the diffusion of pre-selected molecule and other agents into, and out of the converting agent-containing hollow fibers. The purpose of the outer membrane is to hold the bundle of fibers together and not to limit diffusion of reagents, for example, in the case of viable cells: oxygen; nutrients and preselected molecule, into the hollow fibers or the diffusion of waste products, i.e., carbon dioxide, and the pre-selected molecule out of the hollow fibers. In such configurations, the resulting bundles of hollow fibers usually have an external diameter sufficient to permit entrapment by the cartridge of the anchor.

Converting Agent

It is contemplated that a variety of converting agents may be useful in the practice of the invention. Preferred converting agents may include catalysts and most preferred converting agents include biocatalysts that catabolize the pre-selected molecule into one or more other molecules that are less harmful than the pre-selected molecule or are not associated with the disorder. Preferred biocatalysts include, for example, viable cells and enzyme preparations that convert or modify the pre-selected molecule to one or more other molecules that are less harmful than the pre-selected molecule or are not associated with the disorder. Each type of converting agent and factors to optimize their respective activities are discussed in more detail below.

Viable Cells

It is contemplated that a variety of cell types may be used in the practice of the invention. The cells preferably are eukaryotic cells, and more preferably are mammalian cells. Most preferably, the implanted cells are autogeneic in nature, i.e., the implanted cells are derived from the intended recipient. As discussed above, because the cells of the invention may be enclosed in an immunoprivileged environment within a semi-permeable membrane, for example, when the pre-selected molecule has a molecular weight less than 150 kilodaltons, it is contemplated that allogeneic cells, i.e., cells derived from another individual within the same species as the intended recipient, or alternatively xenogeneic cells, i.e., cells derived from a species other than the species of the intended recipient, may be used in the practice of the invention.

The cells incorporated within the device preferably are isolated cells, established cells, or cell lines that catabolize the pre-selected molecule of interest. Such cells or cell lines usually are isolated by standard cell culture and screening procedures well known and thoroughly documented in the art. Reviews discussing such conventional culture and screening procedures include, for example, “Tissue Culture, Methods and Applications” (1973) Kruse and Paterson, Eds., Academic Press, New York, San Francisco, London; “Culture of Animal Cells, A Manual of Basic Technique,” Second Edition (1987) Freshney, Ed., Wiley-Liss, New York, Chichester, Brisbane, Toronto, Singapore; “Cell Biology, A Laboratory Handbook” (1994) Celis, Eds., Academic Press; and “Control of Animal Cell Proliferation” (1985) Boyton and Leffert, Eds., Academic Press.

Although the cells or cell lines of interest preferably are isolated from the recipient and expanded by standard cell culture methodologies prior to implantation, it is contemplated that useful cells or cell lines may be isolated from individuals of the same species other than the intended recipient. Alternatively, useful cells or cell lines may be isolated from individuals belonging to other species, i.e., of porcine, murine, equine, bovine, simian, canine or feline origin.

In addition to the use of naturally occurring cells or cell lines that catabolize the molecule of interest, it is contemplated that cells may be engineered by conventional recombinant DNA methodologies to catabolize a pre-selected molecule or a combination of such molecules. The processes for manipulating, amplifying, and recombining nucleic acids encoding a protein of interest (e.g. a protein that promotes catabolism of the pre-selected molecule) generally are well known in the art and, therefore, are not described in detail herein. Methods for identifying and isolating genes encoding a protein also are well understood, and are described in the patent and other literature.

Briefly, the production of DNAs encoding catabolic proteins of interest is performed using known techniques involving the use of various restriction enzymes which make sequence specific cuts in DNA to produce blunt ends or cohesive ends, DNA ligases, techniques enabling enzymatic addition of sticky ends to blunt-ended DNA, construction of synthetic DNAs by assembly of short or medium length oligonucleotides, cDNA synthesis techniques, polymerase chain reaction (PCR) techniques for amplifying appropriate nucleic acid sequences from libraries, and synthetic probes for isolating genes encoding the protein of interest. Various promoter sequences from bacteria, mammals, or insects to name a few, and other regulatory DNA sequences used in achieving expression, and various types of host cells are also known and available. Conventional transfection techniques, and equally conventional techniques for cloning and subcloning DNA are useful in the practice of this invention and known to those skilled in the art. Various types of vectors may be used such as plasmids and viruses including animal viruses and bacteriophages. The vectors may exploit various marker genes that impart to a successfully transfected cell a detectable phenotypic property that can be used to identify which of a family of clones has successfully incorporated the recombinant DNA of the vector.

DNA encoding the protein of interest may be isolated from libraries of nucleic acids, for example, by colony hybridization procedures such as those described in Sambrook et al. eds. (1989) “Molecular Cloning”, Coldspring Harbor Laboratories Press, NY, and/or by PCR amplification methodologies, such as those disclosed in Innis et al. (1990) “PCR Protocols, A guide to methods and applications”, Academic Press, the disclosures of which are incorporated herein by reference. The nucleic acids encoding the protein of interest, once isolated, may be integrated into an expression vector and transfected into an appropriate host cell for protein expression. Useful prokaryotic host cells include, but are not limited to, E. coli, and B. Subtilis. Useful eukaryotic host cells include, but are not limited to, yeast cells, insect cells, myeloma cells, fibroblast 3T3 cells, monkey kidney or COS cells, Chinese hamster ovary (CHO) cells, mink-lung epithelial cells, human foreskin fibroblast cells, human glioblastoma cells, and teratocarcinoma cells.

The vector additionally may include various sequences to promote correct expression of the recombinant protein, including transcriptional promoter and termination sequences, enhancer sequences, preferred ribosome binding site sequences, preferred mRNA leader sequences, preferred protein processing sequences, preferred signal sequences for protein secretion, and the like. The DNA sequence encoding the protein of interest also may be manipulated to remove potentially inhibiting sequences or to minimize unwanted secondary structure formation.

Expression of the engineered genes in eukaryotic cells is preferably done with cells and cell lines that are easy to transfect, are capable of stably maintaining foreign DNA with an unrearranged sequence, and which have the necessary cellular components for efficient transcription, translation, post-translation modification, and possibly secretion of the protein. In addition, a suitable vector carrying the gene of interest also is necessary. DNA vector design for transfection into mammalian cells should include appropriate sequences to promote expression of the gene of interest as described herein, including appropriate transcription initiation, termination, and enhancer sequences, as well as sequences that enhance translation efficiency, such as the Kozak consensus sequence. Preferred DNA vectors also include a marker gene and means for amplifying the copy number of the gene of interest. A detailed review of the state of the art of the production of mammalian proteins in foreign cells, including useful cells, protein expression-promoting sequences, marker genes, and gene amplification methods, is disclosed in Robbins, P. D. (e.d.) “METHODS IN MOLECULAR MEDICINE: GENE THERAPY PROTOCOLS” (1997) Humana Press, Totowa, N.J.

The various cells, cell lines and DNA sequences that can be used for mammalian cell expression of the pre-selected molecule are well characterized in the art and are readily available. Other promoters, selectable markers, gene amplification methods and cells also may be used to express the proteins of this invention. Particular details of the transfection, and expression protocols are well documented in the art and are understood by those having ordinary skill in the art. Further details on the various technical aspects of each of the steps used in recombinant production of foreign genes in mammalian cell expression systems can be found in a number of texts and laboratory manuals in the art, such as, for example, F. M. Ausubel et al., ed., “Current Protocols in Molecular Biology”, John Wiley & Sons, New York, (1989).

Furthermore, the cartridge may optionally include cells that produce and secrete a desirable molecule such as an anticoagulant or other drug in the circulation. For example, under particular circumstances, for example, during the use of polysulfone hollow fibers, the formation or capture of thrombii on or around the device may affect the flow of blood around the device and/or the diffusion of nutrients or metabolites into or out of the hollow fibers. Under, these circumstances, it is contemplated that a cell type that constitutively produces and secretes an anti-coagulant, for example, tissue plasminogen activator, streptokinase, urokinase, hirudin or the like, into the blood stream also may be included within a hollow fiber. Therefore, the artisan may produce a device containing cells that either on their own or in combination produce an anticoagulant in addition to catabolizing a pre-selected molecule.

By way of example, it is contemplated that a gene encoding the anti-coagulant protein hirudin may be introduced into a host cell by conventional gene transfer methodologies. The local production of hirudin by cells, for example endothelial cells may prove especially attractive in preventing thrombosis at vascular sites. Studies have shown that hirudin is an effective anticoagulant in vivo and is superior to heparin in experimental animal models of thrombosis following arterial injury (Haskel et al. (1991) CIRCULATION 83: 1048-1056; Heras et al. (1990) CIRCULATION 82: 1476-1484). For example, the hirudin encoding gene may be isolated by standard PCR protocols and ligated into a retroviral expression vector, for example pMFG Moloney murine leukemia tumor virus (Dranoffet al. (1993) PROC. NATL. ACAD. SCI. USA 90: 3539-3542) downstream of a nucleic acid sequence encoding a signal sequence for von Willebrand factor (vWF). The vector subsequently may be packaged into φ-crip, an amphotropic, replication defective recombinant retrovirus (Danos et al. (1988) PROC NATL. ACAD. SCI. USA 85: 6460-6464). Endothelial cells, i.e., rabbit endothelial cells or human umbilical vein endothelial cells, or other cells, subsequently may be infected with the recombinant retrovirus, which results in the transfer of the hirudin gene into the genome of the endothelial cell. The transfected cells subsequently constitutively produce and secrete the recombinant hirudin gene product.

Since the transport of oxygen may become a limiting factor for the viability and function of implanted cells, the geometry of the cartridge and any hollow fibers therein must be chosen with care to maintain adequate oxygen delivery. The transport of oxygen from the lumen of the blood vessel to the cells enclosed within the cartridge can occur by diffusion, however, more preferably the transport of oxygen is facilitated by convective transport. When mass transfer occurs primarily by diffusion, studies have shown that, in order to maintain the viability of cells excluded from the blood stream or a blood supply, the cells preferably are located within a critical diffusion distance of about 500 μm, more specifically about 300 μm from the blood supply. For example, direct measurement of the dissolved oxygen levels in mammalian thoracic aortas with oxygen electrodes show that the level of dissolved oxygen in the arterial wall approaches a nadir of 25 mm Hg approximately 300 μm from the blood lumen (Buerk et al. (1982) AM. J. PHYSIOL. 243: H948-H958). Accordingly in such devices, in order to ensure optimal aeration conditions, it is contemplated that the hollow fibers containing the cells have an internal diameter preferably less than about 1000 μm (1.0 mm), and most preferably less than about 500 μm (0.5 mm). It is noted that cells having a low metabolic activity, and therefore low oxygen demand, for example, myoblasts may remain viable in hollow fibers having internal diameters exceeding about 500 μm, however, cell types having a high metabolic activity preferably are entrapped within hollow fibers having internal diameters of about 200 μm. It is contemplated that the optimal cartridge diameter for a pre-selected cell type may be determined without undue experimentation.

In addition to adequate aeration, it is important that the encapsulated cells obtain sufficient amounts of essential nutrients from the blood supply to remain viable. It is believed that oxygen diffusion is the most important aspect in maintaining cell viability and, therefore, once the geometry of a hollow fiber has been optimized for oxygen transport, then the hollow fiber inherently may permit the diffusion and/or convective transport of adequate amounts of nutrients into the lumen of the hollow fiber from the blood stream. Similarly, such a geometry is contemplated also to permit diffusion and/or convective transport of cell metabolites, including, waste products, out of the hollow fiber and into the blood stream.

It is understood, however, that the cell type will depend upon the disease or symptom to be treated. For example, in order to produce a device suitable for treating of dialysis-related amyloidosis, renal proximal tubule cells (RPTC) which have the inherent capacity to uptake and catabolize β₂-microglobulin (β₂M) can be included in the cartridge. Cells having utility in such a device preferably are isolated from either healthy individuals of the same species as the recipient, or from healthy members from other species, i.e., mammals of porcine, bovine, equine or simian origin. Alternatively, a permanent cell line derived from renal proximal tubule tissue, for example LLC-PK1, may be utilized (Sanaka et al, (1989) ASAIO Trans, 35(3): 527-30) in the practice of the invention.

Enzyme Preparation

A variety of enzyme preparations may be useful in the practice of the invention. Enzyme preparations may include pure or substantially pure preparations of enzyme. As used herein, the term “substantially pure” is understood to mean greater than about 60% pure, more preferably greater than about 75% pure and most preferably greater than about 90% pure. Alternatively, crude enzyme preparations may also be useful in the practice of the invention. Crude enzyme preparations may include non viable cells in which enzyme activity is preserved by fixing the cells with a fixative, for example, glutaraldehyde. In general enzymes useful in the practice of the invention can be isolated from plant, microbial, insect, or animal tissues or fluids, or produced in vitro by isolated cells that express and produce the enzyme naturally or after genetic manipulation.

Enzymes may be incorporated into the cartridge in a variety of ways depending on the technical and economical requirements of particular applications. In the most preferred embodiments the enzyme is immobilized within the cartridge so that it remains in place thus minimizing loss via leaching processes. Crude enzyme preparations consisting of non-living cells may be immobilized using physical entrapment systems similar to those used for living cells. Purer enzyme preparations may also be immobilized, both by chemical or physical means. For example, enzymes may be cross-linked to form an enzyme matrix, attached to insoluble matrices through covalent bonds, or cross-linked by multi-functional reagents so they form insoluble enzyme matrices by themselves. Similarly, they may be trapped within the lumen of a semi-permeable membrane if the molecular weight cut-off of such membranes is sufficiently small to prevent passage of the enzyme through the pores. Alternatively, the enzymes may be entrapped in insoluble gel matrices or spun fibers whereby their mobility is retarded. The selection of appropriate immobilization techniques and procedures has been described in detail in the literature, for example as seen in Mosbach K (ed) “Immobilized Enzymes,” METHODS IN ENZYMOLOGY, vol. XLIV, Academic press, NY, 1976.

As an example, the cartridge may include the enzyme phospholipase A2 (PLA2) which metabolizes LDL. In this embodiment, PLA2 is immobilized in the interior of a cartridge which is then implanted according to the device of this invention inside a large blood vessel, for example into the vena cava, and retained by a preimplanted anchor. Circulating LDL in the blood stream enters the cartridge whereupon PLA2 hydrolyzes certain phospholipids thereby converting the molecule. LDL converted accordingly is removed rapidly by the host at rates much faster than removal of unmodified LDL. The extracorporeal use of PLA2 to reduce the level of circulating LDL has been tested in animal models with positive results both in terms of efficacy and safety (see, e.g., Shefer et al. (1993) INT J ARTIF ORGANS 16: 218-28; Labeque et al. (1993) PROC NATL ACAD SCI USA 90: 3476-80). Due to the large molecular weight of LDL (3,500,000 Dalton) the diffusivity of the molecule is very low. Consequently, in practice the cartridge must rely on convection to transport LDL from the bloodstream to the immobilized PLA2 and of PLA2-modified LDL back out of the cartridge into the circulation.

Preparation of the Cartridge

The converting agent may be either pre-loaded into the cartridge prior to implantation or after implantation. When the converting agent comprises viable cells, the cells may be grown in culture in vitro under conventional conditions and then when the requisite number of cells have been attained they can be harvested and introduced into the cartridge for implantation. Alternatively, cells may be introduced into the cartridge at low density and then permitted to multiply in vitro, for example, by means of a commercially available bioreactor. When the converting agent is an enzyme preparation, for example, purified or crude enzyme, either as is or immobilized, then when the requisite amount enzyme preparation has been attained, the enzyme preparation may likewise be introduced into the cartridge pre- or post implantation. It is contemplated, that the final cartridge may contain a variety of different cell types, enzyme preparations or combinations of cells and enzyme preparations to achieve the required blood conditioning effect.

Biocompatability of Anchor and Cartridge

The device of the invention is designed to allow the uncompromised passage of blood around it, and to reduce the possibility of thrombogenic or complement responses elicited by the host against the device. Thus, the size of the device depends upon the size of the blood vessel in which it is to be implanted. For example, the cartridge should preferably be less than 2 cm in diameter if it is to be implanted into a vena cava having a diameter of 4 cm, which leaves about 75% of the cross-sectional surface area of the vessel free to permit blood flow. The device may be adapted to enhance long-term performance, for example, by optimizing blood flow around the device. Such a design, therefore, provides shear levels around the cartridge appropriate to prevent the adhesion of platelets onto the blood contacting surface of the device and/or the formation of thrombus and clot, or stenosis.

Similarly, it is also contemplated that the performance of the device may be enhanced by improving the biocompatibility of all of the device materials that come in contact with blood. For example, the viability and performance of the cells within the cartridge may be enhanced by reducing fibrin and/or platelet deposition on, or thrombus formation around the blood contacting surface of the cartridge. It is contemplated that fibrin and platelet deposition on, or thrombus formation around the blood contacting surface of the cartridge may create additional layers which produce a greater transport resistance for oxygen, thereby limiting cell viability. This problem may be resolved by improving the hemocompatability of the membrane.

In this regard, a number of approaches have been developed to improve hemocompatability of biomaterials placed within the systematic circulation (see, e.g., Ishihara (1993) “Blood compatible polymers”, in BIOMEDICAL APPLICATIONS OF POLYMERIC MATERIALS, Tsuruta T, Hayashi T, Kataoka K, Ishihara K, Kimura Y(eds), CRC Press, Boca Raton, Fla.). These efforts include elimination of protein adsorption by increasing material hydrophilicity, diminishing the blood-material interface by increasing hydrophobicity, inhibiting adhesion and activation of platelets by incorporating microphase separation on the surface of the cartridge, incorporating highly mobile hydrophilic moieties, such as polyethylene oxide, and negative charges that simulate the surface properties of blood vessels, or incorporating biologically active molecules on the surface to inhibit the reaction cascade of biological systems such as the coagulation system. The latter is the most extensively developed approach, whereby heparin can be incorporated into a biomaterial to attain local anti-coagulation activity on the surface of the biomaterial. For example, Duraflo II heparin membranes (Bentley Labs, Baxter Healthcare Corporation, Irvine California) comprise a layer of heparin on the coated surface of membrane which is effective for, at least, several days. See, for example, Hsu (1991) PERFUSION 6:209-219; Tong et al. (1992) ASAIO JOURNAL 38:M702-M706. For example, heparin fragments, prepared from the degradation of heparin in nitrous acid, can be covalently linked by end-point attachment of the heparin to a polyethyleneimine polymer coat (Larm et al. (1983) BIOMAT. MED. DEV. ART ORGANS 11(2&3):161-173, Larssonetal. (1987) ANN N.Y. ACAD. SCI. 516:102-115). This process has been shown to provide effective anticoagulant activity on the surface of biomaterial for several months (Larsson et al. (supra)). It is contemplated that heparinization of the blood contacting surface of the cartridge may minimize fibrin and platelet deposition and/or thrombus formation.

Alternatively, anticoagulants may be delivered continuously into the bloodstream around the device. Anticoagulants can be released either from living cells (as discussed previously) or a drug delivery system (for example, a polymeric sustained release system) incorporated in the cartridge or the anchor. Incorporation of an anti-coagulant delivery system in the cartridge is generally preferable as it can be replenished by replacing the cartridge. On the other hand, anti-coagulant delivery through the anchor may be particularly useful in cases where for long periods of time the anchor may not be accompanied by a cartridge. In either case, the major advantage of local delivery over systemic administration of anti-thrombotic molecules is the much higher efficiency of the former; this way much smaller amounts can be administered reducing greatly the medical risks associated with systemic administration of anti-clotting drugs, as well as much lower costs and substantially reduced technical difficulties. U.S. Pat. No. 5,383,928 discloses that the localized dosage of anti-thrombin agent, D-Phe-Pro-Arg chloromethyl ketone peptide required for complete inhibition of stent thrombosis is 500-fold lower than the intravenous dose required for an equivalent degree of inhibition.

The resulting cartridge subsequently can be implanted together with the anchor into the vasculature of the recipient. Methods for implantation are discussed below.

Implantation of the Device

The device of the invention can be inserted into the vasculature of the host by a variety of non-invasive or minimally invasive surgical procedures. More specifically, it is contemplated that the devices of the invention may be introduced by a variety of catheter-based devices such as those that have been developed for implanting stents and blood clot anti-migration filters into the vasculature.

For example, U.S. Pat. Nos. 3,952,747, 5,147,379, and 5,415,630, and International Patent Application No. PCT/US92/08366, describe catheter-based devices and methods for implanting blood clot anti-migration filters into the vasculature of a recipient. Typically, the catheter-based filter insertion instruments comprise: a carrier for supporting a blood clot anti-migration filter in a collapsed, compact state; an ejector mechanism, usually located within the carrier for ejecting the filter at the pre-selected site; and an elongated, flexible tube connected to the carrier for advancing the carrier along the blood vessel to the pre-selected location. Once introduced to the preferred location in the blood vessel, the filter is ejected from the carrier. When self opening and implanting filters are used, the filter is simply ejected from the carrier, whereupon the filter anchors itself to the wall of the blood vessel. If, however, a filter to be manually opened and anchored is used, then the insertion instrument may contain additional means for opening and anchoring the filter.

Filters typically are inserted through the internal jugular or femoral vein by percutaneous puncture. During percutaneous insertion, and after a conventional cavogram, either the jugular or the femoral vein is punctured with a needle and a guide wire inserted into the vessel through the needle. Then, a combined sheath/dilator unit is pushed into the vein over the guide wire until the end of the sheath is located beyond the implant site. While holding the sheath in place, the dilator and guidewire are removed, leaving the sheath behind. The sheath acts as an access to permit the insertion of the introducer catheter, which contains a carrier holding the filter. The sheath is flushed with sterile heparinized saline to prevent potential thrombus formation within the sheath which may occur during insertion of the introducer catheter. The introducer catheter is advanced into, but not beyond the end of, the sheath until the tip of the filter carrier is positioned adjacent to the implant site. Then, the sheath is retracted onto the introducer catheter until the carrier is completely exposed. Then, the filter is pushed out of the carrier by a pusher mechanism, whereupon the legs of the filter spring outward and engage the inner wall of the blood vessel thereby anchoring the filter in position. It is contemplated that the anchor can be implanted by the skilled practitioner following a similar procedure. Once the anchor has been ejected and anchored in the blood vessel, the cartridge likewise may be introduced via the same catheter into the blood vessel at a position upstream of the anchor. Use of anchor and cartridge elements featuring a complementary locking mechanism would further enable the delivery of the cartridge from either side of the anchor. Then, the introducer catheter can be removed from the vessel through the sheath. Once the introducer catheter has been removed, the sheath also is removed, and the puncture site compressed until homeostasis is achieved.

The procedure for implanting stents follows steps analogous to those described above, especially in the case of self-expanding stents. In the case of stents that do not self-expand, the procedure requires additional steps, as balloon-type catheters typically are used to dilate the contracted stent. Balloons are first dilated to expand the catheter and then are deflated to permit withdrawal of the balloon-type catheter. A variety of stent designs and deployment procedures have been developed and are known to those skilled in the art. Exemplary stent designs and corresponding implantation procedures are disclosed, for example, in U.S. Pat. Nos. 4,655,771; 5,071,407; 5,078,720; 6,113,608; 5,792,172; 5,836,965; 6,113,62; 6,123,723; and 6,136,011.

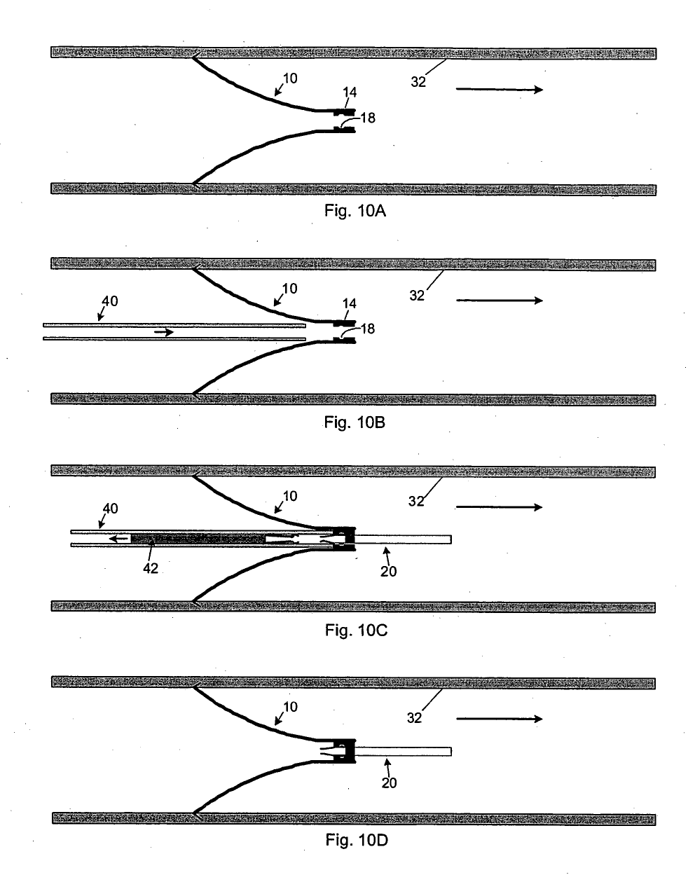

Once immobilized in situ, the cartridge may be introduced into the blood vessel and locked to the immobilized anchor as illustrated in FIG. 10. The direction of blood flow is illustrated by the arrows. FIG. 10A shows anchor 10 immobilized to the inner wall 32 of the blood vessel. The cross-sectional view shows receptacle 14 containing interlocking mechanism 18. FIG. 10B shows the insertion catheter 40 in relation to immobilized anchor 10. FIG. 10C shows cartridge 20 being delivered along catheter 40 via grabbing element 42. Once in place, the grabbing element 42 releases cartridge 20, and the cartridge's locking members extend until the interlocking mechanism on cartridge 20 mates with and engages with the interlocking mechanism 18 of the anchor. Once cartridge 20 is engaged, the grabbing element 42 is withdrawn. Thereafter, the insertion catheter 40 is withdrawn leaving the immobilized anchor 10 and cartridge 20 components of the drug delivery device in place (FIG. 10D). This procedure can be reversed to remove the cartridge in the event of complications or upon termination of therapy, or eventually, to replace the cartridge with a new one containing the same or a different cell type or enzyme for continued and/or modified therapy. Furthermore, the foregoing implantation and/or retrieval procedure is flexible and can be used with a wide variety of anchors and/or cartridges.

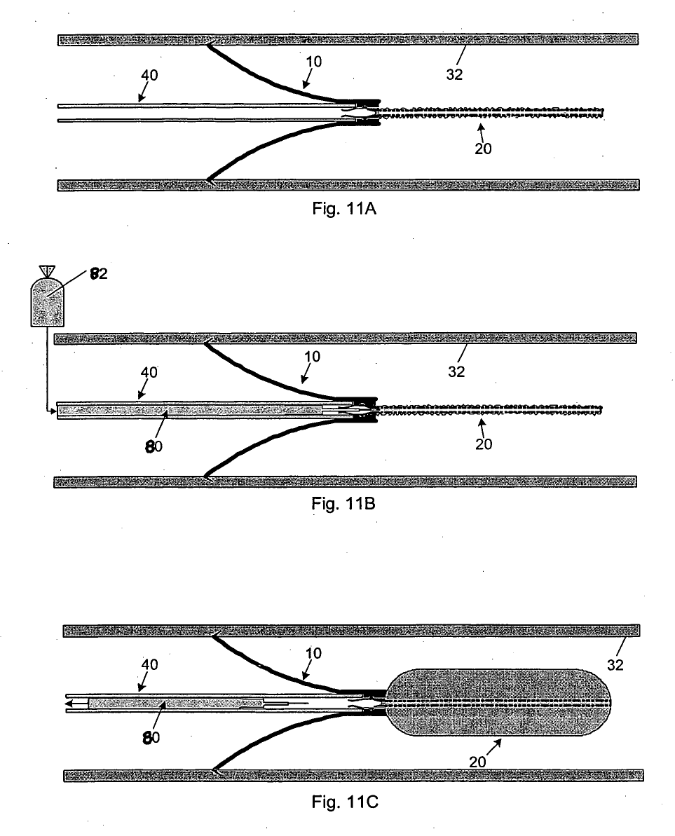

A similar procedure may also be used when the cartridge is empty and is filled with converting agent when immobilized in situ. FIG. 11 illustrates an exemplary protocol for loading a cartridge with converting agent in situ. FIG. 11 illustrates anchor 10 immobilized to an inner wall 32 of a blood vessel, and an empty cartridge 20 engaged by the anchor. Insertion catheter 40 is shown in spatial relation to anchor 10 and cartridge 20. FIG. 11B illustrates a conduit 80 disposed within insertion catheter 40. The conduit has at one end a loading device for introducing cells or enzymes into the cartridge and at the other end it is connected to an extravascular reservoir 82. Extravascular reservoir 82 preferably is at an extracorporeal location. The loading device at the end of conduit 80 may comprise a syringe needle that is capable of piercing, for example, a rubber septum disposed in the cartridge through which drug can be introduced into the cartridge. Gravity or an external pump may be used to deliver the converting agent from extravascular reservoir 82 into cartridge 20. FIG. 11C shows that once cartridge 20 is filled, conduit 80 can be retracted through catheter 40. After withdrawal of conduit 80 catheter 40 can be retracted leaving the device in 'situ.

In an alternative embodiment, the cartridge may be refilled in situ with converting agent once the cartridge has been exhausted. In this approach, a refill catheter or other conduit provides fluid flow communication between the cartridge and an extravascular element (for example, a reservoir, a pump, and/or a vascular access port). Once exhausted, the cartridge can be refilled with converting agent from the extravascular element, for example, an intracorporeal or an extracorporeal element, more preferably an extracorporeal element. It is contemplated that the refill catheter may either be transiently or permanently attached to the cartridge.

It is understood that the preferred location for implantation of the device within the systemic circulation, however, may depend upon the intended use of the device. It is contemplated that the devices may be implanted in situ within an artery or vein. For example, in some situations it is contemplated that it may be desirable to introduce the devices via the femoral or jugular veins and then anchor the anchor at a location within a natural vein, such as, an inferior vena cava, a superior vena cava, a portal vein or a renal vein. Alternatively, the device of the invention may be anchored in a synthetic vein, such as a vein developed from a surgically-developed arteriovenous fistula. Alternatively, the physician may choose to implant the devices at a location upstream or downstream of a natural site of generation or catabolism of the pre-selected molecule. For example, β₂-microglobulin typically is catabolized by the kidney. Accordingly, it may be desirable to introduce and anchor a β₂-microglobulin-catabolizing device in the circulatory system downstream of the kidney to minimize the clearance load imposed on the device. On the other hand, in certain circumstances, it may be desirable to place the device upstream of the kidney to improve its efficiency as the concentrations of β₂-microglobulin are higher which, therefore, result in higher mass transfer rates. It is understood, however, that based upon clinical circumstances, a physician may determine on a case by case basis the optimal mode for introducing the device as well as the optimal location for anchoring the device. Such judgments are contemplated to be within the scope of expertise of the skilled physician.

Practice of the invention will be still more fully understood from the following example, which is presented herein for illustration only and should not be construed as limiting the invention in any way.

### EXAMPLE 1

### Implantation Studies of Intravascular Devices

Studies were performed to test whether an intravascular device can be implanted into a blood vessel without adverse reaction. While these studies did not use an intravascular blood conditioning device, per se, these studies show that it is possible to implant an intravascular device into the blood vessel of a host. Nevertheless, the device itself, as described below, was similar in shape to that shown in FIGS. 3A and 3B.

These studies were conducted by implanting a device into a dog's vena cava through a venotomy using a catheter delivery system. No negative effects due to the device were observed. The animal's health was not compromised for the duration of the study (21 days). Additionally, implantation did not compromise vena cava patency, or patency of other vessels, for the duration of study. Furthermore, the device itself remained intact and stayed at the implantation site (no creeping or migration).

The devices were constructed by combining drug delivery cartridges (i.e., reservoirs) with anchors. The devices were similar to that shown in FIGS. 3A and 3B. In addition, the devices had a conical flow director between the cartridge reservoir and the anchor. Because this experiment focused on the interaction between intravascular implant and host animal, the cartridge was affixed permanently to the anchor rather than through a coupling system. For the same reason, the device was implanted into the animal through a venotomy rather than using a percutaneous delivery system.

The devices were constructed using an ALZET° osmotic minipump, model number 1002, available commercially from ALZA Scientific Products (Mountain View, Calif.), as the model cartridge. The cartridge was affixed to the anchor with a rapid cure ethyl cyanoacrylate adhesive (Insta-Cure 3SI-1, available from BSI, Atascadero, Calif.). The coupling of the cartridge to the anchor was streamlined with a flow director machined out of 0.25 inch diameter PTFE rods. The flow director slid over the head of the anchor and maintained its location through a friction fit. Additionally, the flow director had a generally conical shape with the narrow portion constructed to be located upstream when the device was immobilized in situ and the wide portion constructed to be located downstream when the device was immobilized in situ. This shape allowed the flow director to direct blood flow around the cartridge. The flow director also was machined at the wide end to present a concave surface complementary to a convex surface of the cartridge in order to provide a receptacle for the cartridge and allow for a good fit and seal between the components. The anchor was either a commercial blood clot anti-migration filter (a Greenfield® filter) or a similar straight-limb filter constructed with medical grade 0.015 inch stainless steel (316L) wire. For example, one device was constructed with a 12-F Greenfield® filter as the anchor and a mico-osmotic pump as the cartridge. These two components were interfaced with a teflon flow director.

During construction, the anchor and flow director were sterilized with ethylene oxide prior to affixing the cartridge. The cartridge was purchased sterile. It was filled with a sterile solution or suspension of the agent to be delivered and assembled aseptically under a laminar flow hood. The filled cartridge reservoir then was affixed to the anchor with the sterile instant cure adhesive, and the complete device assembly was placed into a delivery catheter, a sterile PTFE tube with a 5/16 inch inner diameter and a 1/32 inch wall thickness. The size of the catheter was selected so that it would fit easily into the vena cava of the test animals (dogs) while still accommodating the device, allowing the device to glide through it when pushed by a plunger.

Large dogs, weighing approximately 30 kg, were used for the implantation procedure. Prior to surgery, the animals were fasted overnight but provided with water ab libitum. Before surgery, the dogs were given an injection of 0.2 mg/kg Butaphenol, 0.05 mg/kg Acepromazine, and 0.01 mg/kg Glycopyrollate as proanesthesia. The animals then were anesthetized via intravenous administration of 200 mg pentothal, intubated, and maintained under anesthesia with 2% isofluorane (balance oxygen).

After the vena cava was exposed, the renal arteries and veins were isolated and occluded. Immediately, the vena cava was cross-clamped to prevent flow and a partial venotomy was performed. The delivery catheter containing the device was inserted into the vena cava through the opening. The device was placed such that the cartridge was facing downstream. Subsequently, the device was pushed inside the catheter with the aid of a plunger. Following its exit from the catheter, the device's anchor expanded, engaging the vessel wall. Then, the plunger and catheter were then withdrawn, leaving the device implanted in situ. The vena cava section then was closed with 5.0 proline sutures. The blood vessel clamps and ties were removed and, after careful inspection for bleeding, the abdominal cavity was closed using a three-layer closure with 2-0 Vicryl suture. Post-operatively, animals were given 0.02 mg Bupemex for pain relief as well as 800 mg of Bacterim, an antibiotic, twice daily to prevent infection. After recovery, the animals were returned to their cages. The life of the ALZET® pump used in this study (21 days) provided the upper limit for the implantation period.

Following implantation, vena cava patency was verified by performing fluoroscopies at fixed time intervals. At the end of the experiment, the animal was euthanized. The vena cava was removed along with the implanted device, rinsed, and sectioned longitudinally to reveal the implant for evaluation of the host-implant interaction. To evaluate the extent of thrombus formation as a result of the device presence in the intravascular space, the heart and lungs were removed and sectioned to determine if thrombi had lodged into blood vessels and occluded them. Heart and lung samples also were collected along with samples of cava, liver, and kidney tissue for subsequent analysis for the presence of agents infused through the implanted drug delivery cartridge.

Blood flow through the vena cava was not compromised by the intravascular implant. Fluoroscopic images taken at 18 days post implantation, the last fluoroscopy performed prior to study termination at 21 days, revealed that blood flow was uncompromised. Flowing blood registered around the drug delivery cartridge reservoir, which appeared symmetrically in the center of the vessel. This unoccluded flow was seen despite the fact that the diameter of the cava (approximately 10 mm) was only slightly larger that the diameter of the implant (approximately 6 mm). A human vena cava is larger, typically larger than about 20 mm in diameter, so patency in humans should be less of a concern. In addition, this fluoroscopic analysis indicated that the device blood flow was not compromised seriously even in the interior of the anchor and that the device retained its integrity.

After the animal was euthanized at 21 days, the following observations were made. There was no compromise of the cava wall, no inflammation, and no migration of the device. Also, a portion of the anchor limbs were incorporated into the vessel endothelium, but the cava lumen was clean and free of any adhesions. There was some clotting at the device itself, primarily around areas of stagnant flow (for example between the anchor limbs), but, based on the autopsy, their presence was limited to that area. Finally, there were no signs of clotting or thrombi in any of the analyzed tissues, including the vena cava, heart, and lungs.

Additionally, the strength of engagement between anchor and cava wall was analyzed. During harvesting and longitudinal sectioning of the vena cava to observe the device and cava, all 6 limbs of the anchor were kept engaged to the cava wall. Accordingly, a spring-based force meter was used to pull the anchor apart from the cava wall. The force measured prior to separation exceeded 2 lb_{f} or 10 N. It is contemplated that a measured engagement force would be even larger if the vena cava was unsectioned.

### EXAMPLE 2

### Flow Studies

The shape of each component of the implantable device preferably is optimized to minimize the degree of interaction between the device and the blood. If stagnant flows and vortices can be reduced or eliminated in the intravascular space in the vicinity of the device, then individual components of blood, for example, circulating platelets, may be prevented from collecting around the device. Furthermore, the residence time of such blood components in contact with the device may be shortened thereby substantially decreasing the potential for clotting. By way of illustration, at a typical flow rate of 2 L/min in an inferior vena cava having a diameter of 2.5 cm, the mean linear velocity of blood is estimated to be 21.3 cm/sec. Accordingly, it is estimated that it would take half a second for blood to flow over a 10 cm long implant. However, the introduction of an implant of substantial size into the vascular space may disturb blood flow considerably and generate areas with eddies and flow stagnation (such areas have been recognized as prone to clotting). It is possible to minimize flow disturbances by streamlining the shape of the implant to yield shapes commonly considered as “aerodynamic.”

The effect of various implant shapes can be visualized using a model flow system that simulates the fluid dynamics of a vena cava containing an implant anchored in the vessel lumen. In such a model, transparent Tygon tubing can be used to simulate a human vena cava. After a test implant is positioned inside the Tygon tubing, water at room temperature is pumped through the tubing via a peristaltic pump. The flow rate can be controlled so as to achieve fluid dynamic similarity between the model system and a human vena cava (i.e., the Reynolds number in the model system is similar to that calculated for blood flowing inside a human vena cava). Fluid flow can be visualized by introducing a colored dye into the tubing, upstream from the implant model. Dye streamlines reveal the nature of the fluid flow for a particular implant model, which can be recorded with a tripod-mounted motion camera.

By implanting test devices comprising a model cartridge of a polypropylene 0.25 inch diameter rod machined to a shape of interest affixed to a model anchor (for example, a 12F Greenfield® filter) into such a model system, it was found that rounding of the edges of the model cartridge was useful to minimize eddies and areas of stagnant flow. Based on this type of study, the degree of rounding required at the front or upstream end of the model cartridge was not as important as that required at the tail or downstream end of the model cartridge. A conical shaped flow director with a radial profile and radius similar to the radius of the polypropylene rod was sufficient to provide a preferred'shape at the front end. A sharper-shaped tail was helpful in minimizing the formation of a turbulent wake at the rear of the model cartridges. The development of wake was found to be dependent on the relative diameter of the model cartridge and the model vena cava. Where the implant cartridge was less than a third of the diameter of the tubing, it was found that a sloping tail design with the tail extending for a distance approximately equal to two diameters of the model cartridge's main body could be sufficient to eliminate wake formation. In contrast, if the tail end of the model cartridge was not shaped (for example, the model cartridge had a pure cylindrical shape), a wake with two symmetrical eddies could be formed. Based on studies of this type, the cartridge shape preferably includes a rounded or sloping tail design extending to an apex, where the distance from the body of the cartridge to the apex of the tail is equivalent to approximately one to approximately three diameter lengths of the body of the cartridge.

### EXAMPLE 3

### Intravascular β₂-Microglobulin Catabolizing Device for Treating Amyloidosis

In healthy humans, the plasma concentration of β₂-microglobulin (β₂M) usually is about 1-2 mg/L. Accordingly, plasma concentrations of β₂M above about 2 mg/L may be considered to be elevated. However, the concentration of β₂M in plasma may rise to above 50 mg/L in individuals with no residual renal function (Revillard et al. (1988) CONTRIB. NEPHROL. 62: 44-53). As of yet, the effects of β₂M are not fully realized but there is strong evidence that high levels of plasma β₂M are associated with amyloid deposits in joint tissues and, frequently lead to the development of debilitating arthritis. Even though the kinetics of β₂M generation and clearance are yet to be fully elucidated, there is strong evidence that certain pathological conditions as well as extracorporeal treatment with low flux cellulosic membranes exacerbate the disorder. Replacement of such hemodialysis membranes with synthetic high-flux membranes of improved biocompatibility can aid in the removal of plasma β₂M whether by means of adsorption onto, or, dialysis and filtration through the membrane.

Extracorporeal treatment with biocompatible high-flux hemodialysis membranes has shown only modest clinical benefits, primarily because at best it can remove only 50% of β₂M production (Odell (1991) supra). The discontinuity of extracorporeal dialytic treatment is the main reason behind the modest β₂M reduction achieved with modern high flux dialyzers. Indeed, even though such dialyzers are characterized by sufficiently high rates of β₂M clearance resulting in substantial β₂M drop while the patient is on dialysis, plasma concentrations start increasing as soon as the dialytic treatment is terminated. During this type of treatment, plasma concentrations of β₂M typically drop to; as low as 29 mg/L after about three hours of dialysis.

Plasma levels, however, start rebounding immediately upon completion of dialysis to reach levels of about 35-40 mg/L. Furthermore, because the amount of β₂M removed during such an extracorporeal treatment correlates positively with β₂M concentration, the effectiveness by which dialysis treatment removes β₂M is reduced as plasma β₂M is reduced.

The device of the invention may be used to treat individuals with elevated levels of plasma β₂M in a continuous fashion over a prolonged period of time, for example, a period exceeding one month and more preferably three months. The converting agent to remove the circulating β₂M molecules can comprise proximal tubule cells (PTC), the same type of cells responsible for β₂M catabolism in vivo. In the healthy kidney, β₂M is filtered through the glomerulus and then taken up and catabolized by the epithelial cells lining the proximal portion of the tubule. Under normal conditions, the rate of β₂M removal is comparable to the glomerular filtration rate (GFR) and β₂M plasma levels are inversely related to GFR indicating that β₂M reabsorption and catabolism constitute rapid processes. Consequently, the rate of β₂M processing by PTCs is similar to the rate of glomerular β₂M filtration, and more likely exceeds it considerably.

Assuming that viable PTCs included as the converting agent in a device of the invention maintain their normal in vivo β₂M catabolic activity, a plasma β₂M level of 10-20 mg/L (approximately 10-fold higher than that in normal subjects) may be achieved by a device comprising one tenth, or less, of the total number of PTCs typical for a healthy adult. Because the total number of PTCs in the kidneys of a healthy adult typically does not exceed 2×10¹⁰ cells (approximately 5-10 ml of tissue), an intravascular blood conditioning device incorporating just 2 mL of PTC tissue could yield stable levels of circulating β₂M that provide substantial therapeutic benefits and are considerably lower than those achieved by extracorporeal therapy with hemodialysis/filtration membranes. Furthermore, a device of the present invention could be utilized to reduce plasma β₂M levels in patients with chronic renal disease who have diminishing renal clearance but do not yet meet the criteria for dialysis.

A PTC tissue volume of 2 mL or less can be accommodated into a blood conditioning device that could be delivered, for example, in the vena cava with the aid of a catheter system. The PTCs may be loaded as dense hydrogel-cell suspension, for example autologous fibrin glue-cell suspension, into a hollow fiber approximately 10 cm long and 3.5 mm wide (having an internal diameter of about 3 mm) defined by a semi-permeable ultrafiltration membrane, for example, made of polyacrylonitrile, polypropylene, or polysulfone. Use of a highly permeable membrane and implantation of the hollow fiber into a high flow environment, such as a vena cava, at a slight angle to the blood flow can optimize mass transport into and out of the hollow fiber and ensure adequate cell oxygenation. The PTCs can be derived from cell lines, isolated from xenogeneic or allogeneic kidneys, or more preferably, developed from autologous sources using stem cell technologies.

Accordingly, an anchor comprising a head and metallic filaments terminating in hooks, for example, the anchor element depicted in FIG. 4 may be implanted with the aid of a catheter into the vena cava of a patient. Isolated and/or cultured viable renal proximal tubule cells are propagated in vitro in roller bottle culture until the desired total cell number, for example, about 10⁹ cells is reached. The cells then are trypsinized, and the resulting cell suspension spun down to be resuspended into a hydrogel. Once resuspended, the cells are introduced into a cartridge, for example, as depicted in FIG. 6A, whereby the cartridge incorporates a locking mechanism that engages a complementary locking mechanism in the anchor. Following induction of hydrogel formation, for example, by diffusion of thrombin into a cartridge containing a cell suspension in fibrinogen, the cartridge then is inserted into the vessel with the pre-immobilized anchor, optionally by the same catheter system, for example, as shown in FIG. 10. Following introduction, the cartridge locking mechanism is aligned with and engaged by the anchor locking mechanism. The introduction catheter then is retrieved leaving the device in situ, whereupon the viable cells take up and catabolize circulating β₂M. As a result, such a device may reduce the concentration of β₂-microglobulin in the bloodstream thereby ameliorating the symptoms of amyloidosis.

### INCORPORATION BY REFERENCE

The disclosures of each of the patent documents and scientific articles identified herein are expressly incorporated by reference herein.

### Other Embodiments

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

Other embodiments of the invention are within the following claims.

What is claimed is:

1. An implantable, intravascular device for treating a disorder associated with the presence of a pre-selected molecule in the blood stream of an animal, the device comprising:
(a) an anchor immobilizable to an inner wall of an intact blood vessel which, when immobilized in the vessel, permits blood in the vessel to pass therethrough; and
(b) a cartridge containing a converting agent, wherein the cartridge when introduced into the blood vessel is retained by the anchor and permits blood in the vessel to pass therethrough and wherein the converting agent is substantially retained within the cartridge during operation and is capable of converting or modifying within the cartridge the pre-selected molecule that enters the cartridge from the blood stream so as to remove the pre-selected molecule from the blood stream.

2. The device of claim 1, wherein the anchor comprises at least one element biased in a radially outward direction when immobilized in the blood vessel.

3. The device of claim 1, wherein the anchor is a stent.

4. The device of claim 1, wherein the anchor comprises an outwardly extending barb.

5. The device of claim 4, wherein the anchor comprises a head and a plurality of barbed filaments attached by one end to the head.

6. The device of claim 4, wherein the anchor is an embolism anti-migration filter.

7. The device of claim 1, wherein the anchor comprises a receptacle for receiving the cartridge.

8. The device of claim 7, wherein the anchor further comprises an interlocking mechanism that engages a reciprocal interlocking mechanism of the cartridge for locking the anchor to the cartridge.

9. The device of claim 1, wherein the cartridge comprises at least one hollow fiber.

10. The device of claim 9, wherein the hollow fiber is defined at least in part by a semi-permeable membrane.

11. The device of claim 10, wherein the semi-permeable membrane defines pores of a size sufficient to permit passage of the pre-selected molecule therethrough.

12. The device of claim 11, wherein the pores permit passage therethrough of a pre-selected molecule smaller than 150 kD.

13. The device of claim 10, wherein the semi-permeable membrane comprises a material selected from the group consisting of polyvinylchloride, polyvinylidene fluoride, polyurethane isocyanate, alginate, cellulose, cellulose acetate, cellulose diacetate, cellulose triacetate, cellulose nitrate, polyarylate, polycarbonate, polysulfone, polystyrene, polyurethane, polyvinyl alcohol, polyacrylonitrile, polyamide, polyimide, polyacrylonitrite, polymethylmethacrylate, polyethylene oxide, polytetrafluorthylene, and mixtures thereof

14. The device of claim 1, wherein the converting agent is a biocatalyst.

15. The device of claim 14, wherein the biocatalyst is a viable cell or an enzyme.

16. The device of claim 15, wherein the viable cell is a eukaryotic cell.

17. The device of claim 16, wherein the eukaryotic cell is a mammalian cell.

18. The device of claim 14, wherein the biocatalyst catabolizes the pre-selected molecule.

19. The device of claim 15, wherein the enzyme is a purified enzyme preparation.

20. The device of claim 15, wherein the enzyme is immobilized on a solid support.

21. The device of claim 1, wherein the pre-selected molecule is harmful to the animal.

22. The device of claim 21, wherein the pre-selected molecule is selected from the group consisting of β₂-microglobulin, lipoprotein, and bilirubin.

23. A cartridge for implantation into a lumen of a blood vessel for treating a disorder associated with the presence of a pre-selected molecule in the blood stream of an animal, the cartridge comprising:
a wall defining an inner volume and defining a plurality of pores passing therethrough dimensioned to permit entry of the pre-selected molecule into the inner volume; and
a converting agent disposed within the inner volume and capable of converting or modifying the pre-selected molecule within the cartridge, wherein the converting agent is substantially retained within the cartridge during operation
and wherein the cartridge, when introduced into a blood vessel, permits blood to pass through the vessel and removes the pre-selected molecule from the blood stream.

24. The cartridge of claim 23, further comprising an interlocking mechanism that engages a reciprocal interlocking mechanism of an anchor immobilizable to an inner wall of the blood vessel for locking the cartridge to the anchor.

25. The cartridge of claim 23, wherein the cartridge comprises a plurality of hollow fibers.

26. The cartridge of claim 23, wherein the pores are dimensioned to permit passage of the pre-selected molecule therethrough but insufficient to permit passage of the converting agent therethrough.

27. The cartridge of claim 23, further comprising an internal diameter of less than about 1000 μm.

28. The cartridge of claim 27, further comprising an internal diameter of less than about 500 μm.

29. The cartridge of claim 23, wherein the converting agent comprises a viable cell or an enzyme.

30. The cartridge of claim 29, wherein the viable cell is a mammalian cell.

31. The cartridge of claim 29, wherein the enzyme is a purified enzyme preparation.

32. The cartridge of claim 29, wherein the enzyme is immobilized on a solid support.

33.-40. (canceled)
